(19) United States
(12) Patent Application Publication
(10) Pub. No.: US 2026/0273091 A1
(43) Pub. Date: Sep. 17, 2026

(54) COLON-TARGETED DELIVERY SYSTEMS AND METHODS OF USE

(71) Applicant: University of Massachusetts, Westborough, MA (US)

(72) Inventors: Gang Han, Shrewsbury, MA (US); Yang Zhao, Tianjin (CN)

(21) Appl. No.: 19/566,864

(22) Filed: Mar. 13, 2026

Related U.S. Application Data
(60) Provisional application No. 63/772,253, filed on Mar. 14, 2025.

(51) Int. Cl. A61K 47/69; A61K 31/58; A61K 47/61; A61P 1/00

(52) U.S. Cl. CPC A61K 47/6923; A61K 31/58; A61K 47/61; A61K 47/6929; A61P 1/00

(57) ABSTRACT

Described herein are colon-targeted delivery systems comprising a mesoporous silica nanoparticle (MSN), loaded with at least one bioactive agent, and encapsulated with inulin. Also provided are methods using the colon-targeted delivery systems to treat a colonic disease or disorder, e.g., inflammatory bowel disease.

### CLAIM OF PRIORITY

This application claims the benefit of U.S. Provisional Application Ser. No. 63/772,253, filed on Mar. 14, 2025. The entire contents of the foregoing are incorporated herein by reference.

### TECHNICAL FIELD

Described herein are colon-targeted delivery systems comprising a mesoporous silica nanoparticle (MSN), loaded with at least one bioactive agent, and encapsulated with inulin. Also provided are methods using the colon-targeted delivery systems to treat a colonic disease or disorder, e.g., inflammatory bowel disease.

### BACKGROUND

Inflammatory bowel disease (IBD), including ulcerative colitis and Crohn's disease, is an incurable chronic inflammatory colorectal disease that afflicts millions worldwide. Individuals with IBD are at increased risk for several malignancies originating in the intestine, such as colorectal cancer.

### SUMMARY

Conventional therapy of bowel diseases has been plagued by poor drug bioavailability and inevitable side-effects. Moreover, these conventional drugs are typically absorbed in the upper gastrointestinal tract (GIT) before they reach the site of disease in the colon, thus making their therapeutic efficacy far from satisfactory. Targeted drug delivery directly into the colon may minimize systemic side effects while providing effective local treatment for a variety of bowel diseases such as colorectal cancer, Crohn's disease, diverticulitis, irritable bowel syndrome (IBS), microscopic colitis, ulcerative colitis and for local treatment of colonic pathologies, including colonic polyps. As such, there is a need in the art for colon-targeted delivery systems.

Provided herein are compositions comprising a mesoporous silica nanoparticle (MSN) and inulin conjugated to the surface of the MSN. In some embodiments, at least one bioactive agent is loaded into the MSN. In some embodiments, at least two bioactive agents are loaded into the MSN. In some embodiments, the at least one bioactive agent or the at least two bioactive agents is/are selected from the group consisting of a small molecule, an organometallic compound, a vaccine, a diagnostic agent, a polynucleotide, a peptide, and a polypeptide. In some embodiments, the inulin conjugated to the surface of the MSN comprises at least one glycosidic bond. In some embodiments, the inulin conjugated to the surface of the MSN is sensitive to enzymatic degradation. In some embodiments, the inulin conjugated to the surface of the MSN is sensitive to enzymatic degradation by inulinase. In some embodiments, the inulin conjugated to the surface of the MSN comprises carboxymethyl inulin (CMI). In some embodiments, the MSN comprises at least one pore on its surface. In some embodiments, the at least one pore comprises a diameter ranging from about 2 nm to about 50 nm. In some embodiments, the composition comprises a particle size ranging from about 100 nm to about 500 nm in diameter. In some embodiments, the composition comprises a particle size of about 275 nm in diameter. In some embodiments, the composition comprises a zeta potential ranging from about −20 mV to about −10 mV.

Additionally, provided herein are pharmaceutical compositions. In some embodiments, a pharmaceutical composition of the present disclosure comprises at least one of the compositions disclosed herein (e.g., a colon-targeted delivery system) and at least one pharmaceutically accepted carrier. In some embodiments, a pharmaceutical composition of the present disclosure comprises an oral formulation. In some embodiments, the oral formulation is selected from the group consisting of tablets, pills, capsules, powders, granules, solutions or suspensions.

Further provided herein are methods of treating a colonic disease or disorder in a subject in need thereof. In some embodiments, a method of treating a colonic disease or disorder in a subject in need thereof comprises administering a therapeutically effective amount of at least one of the compositions disclosed herein (e.g., a colon-targeted delivery system) and/or at least one of the pharmaceutical compositions disclosed herein. In some embodiments, a method disclosed herein provides that less than about 10% of the bioactive material is released from the composition in the subject's upper GI track after administration. In some embodiments, a method disclosed herein provides that more than about 60% of the bioactive material is released from the composition in the subject's colon after administration. In some embodiments, a method of treating a colonic disease or disorder in a subject in need thereof comprises administering a therapeutically effective amount of at least one of the compositions disclosed herein (e.g., a colon-targeted delivery system) and/or at least one of the pharmaceutical compositions disclosed herein in combination with at least one biological agent.

Also provided herein are methods of modulating composition of a gut microbiome in a subject in need thereof. In some embodiments, a method of modulating composition of a gut microbiome in a subject in need thereof comprises administering to the subject in need thereof a therapeutically effective amount of at least one of the compositions disclosed herein (e.g., a colon-targeted delivery system) and/or at least one of the pharmaceutical compositions disclosed herein. In some embodiments, at least one bacteria in fecal microbiota of the subject is increased following the administration of a therapeutically effective amount of at least one of the compositions disclosed herein (e.g., a colon-targeted delivery system) and/or at least one of the pharmaceutical compositions disclosed herein. In some embodiments, the at least one bacteria in fecal microbiota of the subject that is increased is selected from the group consisting of Firmicutes, Bacteroidetes, Verrucomicrobia, Alloprevotella, Lactobacillus, Akkermansia, Prevotellaceae, and Proteobacteria phyla. In some embodiments, the amount of Alloprevotella, Lactobacillus, Akkermansia, Prevotellaceae, or any combination thereof is increased in fecal microbiota of the subject following administration of a therapeutically effective amount of at least one of the compositions disclosed herein (e.g., a colon-targeted delivery system) and/or at least one of the pharmaceutical compositions disclosed herein.

A subject in need thereof of any of the methods provided herein is suspected of having or has been diagnosed as having a colonic disease or disorder. In some embodiments, the colonic disease or disorder is selected from the group consisting of colorectal cancer, colonic polyps, Crohn's disease, diverticulitis, inflammatory bowel disease (IBD), irritable bowel syndrome (IBS), microscopic colitis, and ulcerative colitis (UC). In some embodiments, the colonic disease or disorder is IBD.

Provided herein are also kits for use in any of the methods disclosed herein, wherein the kits can comprise at least one of the compositions disclosed herein (e.g., a colon-targeted delivery system) and/or at least one of the pharmaceutical compositions disclosed herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A and 1B are schematic illustrations of the fabrication and mechanisms of the nanoparticles for colon-targeted therapy of IBD. FIG. 1A: A schematic showing the composition and engineering of the prebiotic nanoparticles Bud@MS-IN NPs. FIG. 1B: Oral delivery of Bud@MSN-IN NPs showed desirable therapeutic effects on colitis mice by selectively releasing the preloaded drug upon encountering inulinase which is presented solely in the colon. Meanwhile, as a prebiotic, inulin regulated the intestinal flora and promoted the recovery of intestinal epithelial functions, fostering the elimination of inflammation.

FIGS. 2A-2F show the preparation and characterization of the nanoparticles. FIG. 2A: Representative DLS analysis and TEM image of Bud@MSN-IN NPs, Scale bar: 100 nm. FIG. 2B: Zeta potential values of MSN, MSN-NH₂, CMI and Bud@MSN-IN NPs. FT-IR spectra (FIG. 2C) and N₂ adsorption/desorption isotherms distribution (FIG. 2D) of MSN, MSN-NH₂ and Bud@MSN-IN NPs. FIG. 2E: TGA results of MSN, MSN-NH₂, and MSN-IN NPs. FIG. 2F: UV-vis absorbance spectra (inner: appearance photo) of Inulin, CMI, MSN-NH₂, and MSN-IN NPs.

FIGS. 3A-3H demonstrate the colon-targeting performance of the nanoparticles. FIG. 3A: In vitro release kinetics of Bud from Bud@MSN-IN NPs under various pH levels and enzymatic media. FIG. 3B: In vitro release kinetics of Bud from Bud@MSN, Bud@MSN-IN and Bud@MSN-OA NPs under simulated gastrointestinal conditions (SGF, simulated gastric fluid; SIF, simulated intestinal fluid; SCF, simulated colonic fluid). Qualitative analysis of (FIG. 3C) and representative images from (FIG. 3D) CLSM imaging of Caco-2 cells incubated with RhB@MSN-IN NPs (200 μg/mL) and RhB@MSN-OA NPs (200 μg/mL) in presence or absence of inulinase (5 mg/mL) for 18 h (i, RhB@MSN-IN; ii, RhB@MSN-IN+inulinase; iii, RhB@MSN-OA; iv, RhB@MSN-OA+inulinase). The fluorescence intensity of RhB in each group was quantitative analyzed using Image J software (FIG. 3C). Cell nuclei were stained with DAPI (blue). scale bars, 50 μm. FIG. 3E: Viability of HT-29 cells after 24 h incubated with different concentrations of Bud suspension or Bud@MSN-IN NPs in presence or absence of inulinase (5 mg/mL). Qualitative analysis of (FIG. 3F) and representative ex vivo fluorescence images (FIG. 3G) illustrating RhB@MSN-IN NPs distribution in the colon of mice with or without colitis after 6 h of oral administration. Qualitative analysis of fluorescence was assessed in the colon of each group (FIG. 3F). FIG. 3H: Representative CLSM images of colon tissue in colitis mice at 6 h post oral administration of RhB@MSN-IN NPs. Scale bars, 100 μm. Data are presented as mean±SD (n=3). *P<0.05, **P<0.01, ***P<0.001, ****P<0.0001. One-way ANOVA followed by Tukey's HSD multiple comparison post hoc test.

FIGS. 4A-4I show application of the nanoparticles for colon-targeted therapy of IBD. FIG. 4A: C57-BL/6 mice were provided with water or 3% DSS-containing water for 5 days, and mice were orally administered PBS or Bud sus (0.6 mg/kg), MSN-IN (equivalent mass of CMI as in Bud@MSN-IN), or Bud@MSN-IN (equivalent mass of Bud as in Bud sus) daily. FIG. 4B: Daily bodyweight changes were monitored in each group for 10 days. On day 10, animals were euthanized and colon length was determined (FIG. 4C). Shown are (FIG. 4D) representative colon images from each group (i, control; ii, colitis; iii, Bud sus; iv, MSN-IN; v, Bud@MSN-IN); scale bars, 50 mm, and (FIG. 4E) representative images of hematoxylin and eosin (H&E)-stained colonic tissues in the five groups; scale bars, 100 μm. MPO activity was measured (FIG. 4F) and levels of the pro-inflammatory cytokines IFN-γ (FIG. 4G), IL-6 (FIG. 4H), and IL-1B (FIG. 4I) in colon samples from the different groups were determined by ELISA. Data are presented as mean±SD (n=3). *P<0.05, **P<0.01, ***P<0.001, ****P<0.0001 versus DSS-induced colitis group; #P<0.05, ##P<0.01, ###P<0.001, ####P<0.0001 versus Bud@MSN-IN group; analyzed by one-way (FIGS. 4C, 4F-4I) or two-way (FIG. 4B) ANOVA with Tukey's HSD multiple comparison post hoc test.

FIGS. 5A-5C depict nanoparticles protection of the colonic epithelium. FIG. 5A: Representative images from the immunofluorescence analysis for the ZO-1 and Occludin-1 expression level in the colon tissues of mice in the different groups (control; colitis; Bud sus; MSN-IN; Bud@MSN-IN). Nuclei were stained with DAPI (scale bars, 100 μm). FIG. 5B: Intestinal barrier functions were assessed in these mice by oral gavage of 4 MW FITC-dextran on day 10, followed by measuring the FITC-dextran signal in blood after 4 h. FIG. 5C: The quantification of FITC fluorescence intensity in FIG. 5B. Data are presented as mean±SD (n=3). *P<0.05, **P<0.01, ***P<0.001, ****P<0.0001 versus DSS-induced colitis group; #P<0.05, ##P<0.01, ###P<0.001, ####P<0.0001 versus Bud@MSN-IN group. Data was analyzed by one-way ANOVA with Tukey's HSD multiple comparison post hoc test.

FIGS. 6A-6H depict modulation of gut microbiome by the nanoparticles. FIG. 6A: Multiple sample PCoA analysis of microbial species abundance using the different treatments as the grouping variable, based on the Bray-Curtis distance. FIG. 6B: NMDS plot illustrating the gut microbiome β-diversity. FIG. 6C: The multiple sample similarity tree. FIG. 6D: The changes of phylum level in fecal microbiota composition. Phylum level taxonomy is presented as percentage of total sequences. FIG. 6E: Heatmap of the relative abundance of genus-level taxa (rows) for each mouse (columns). The abundance is shown as relative percentage. Relative abundance of selected taxa at the genus-level (FIG. 6F: Escherichia shigella; FIG. 6G: Alistipes; and FIG. 6H: Lactobacillus) are shown. Data are presented as mean±SD (n=4). *P<0.05, **P<0.01, ***P<0.001, ****P<0.0001 versus DSS-induced colitis group; #P<0.05, ##P<0.01, ###P<0.001, ####P<0.0001 versus Bud@MSN-IN group. Data was analyzed by one-way ANOVA with Tukey's HSD multiple comparison post hoc test.

FIGS. 7A-7C depict a biocompatibility analysis of oral nanoparticles in vivo. Hematological analysis (FIG. 7A) and quantification of biochemical markers (FIG. 7B) relevant to liver/kidney functions were performed. FIG. 7C: Representative photomicrographs of H&E-stained sections of the major organs (heart, liver, spleen, lung, and kidney) of the different groups are shown, Scale bars, 100 μm. Data are presented as mean±SD (n=5 biologically independent samples for A and n=3 biologically independent samples for B). Statistical analysis was assessed using the one-way ANOVA test. A value of P<0.05 was considered statistically significant.

FIG. 8 is a representative TEM image of MSN NPs, Scale bar: 100 nm.

FIG. 9 is a representative DLS analysis of MSN NPs.

FIG. 10 depicts FT-IR spectrums of Inulin and CMI.

FIGS. 11A and 11B show the ¹H NMR spectra of (FIG. 11A) inulin and

(FIG. 11B) carboxymethyl inulin. The arrow indicates the chemical shift position of carboxymethyl groups attached to inulin.

FIGS. 12A-12C are representative photos of the freeze-dried powder of the nanomaterials (FIG. 12A) MSN, (FIG. 12B) MSN-IN and (FIG. 12C) Bud@MSN-IN.

FIG. 13 depicts a particle size analysis of Bud@MSN-IN NPs in water for 7 days. Data are presented as mean±SD (n=3).

FIG. 14 shows the pore volume distribution of MSN, MSN-NH₂ and Bud@MSN-IN NPs.

FIG. 15 depicts the standard curve of budesonide in methanol showing linearity.

FIG. 16 depicts viability of HT-29 cells incubated with different concentrations of MSN-IN NPs or Bud@MSN-IN NPs for 24 h. Data are presented as mean±SD (n=3).

FIG. 17 shows representative ex vivo fluorescence images illustrating RhB@MSN-IN NPs distribution in the major organs (liver, lung, heart, spleen, kidney) of mice with or without colitis after 6 h of oral administration.

FIGS. 18A and 18B depict (FIG. 18A) typical fluorescence images of the colonic tissue from healthy mice separately treated with free RhB (left) or RhB@MSN-IN NPs (right). FIG. 18B shows the qualitative analysis of fluorescence in FIG. 18A. Data are presented as mean±SD (n=3). *P<0.05.

FIG. 19 shows the biodistribution of budesonide at 24 h post administration of Bud suspension or Bud@MSN-IN NPs in colitis mice. ‘nd’ stands for not detected. Data are presented as mean±SD (n=3). *P<0.05, **P<0.01, ***P<0.001, ****P<0.0001. Statistical analysis was assessed using the one-way ANOVA test.

FIG. 20 shows disease activity index (DAI) of control, colitis, Bud sus, MSN-IN and Bud@MSN-IN NPs treated groups during the whole experimental period. Data are presented as mean±SD (n=3). *P<0.05, **P<0.01, ***P<0.001, ****P<0.0001 versus DSS-induced colitis group; ns=No statistical difference. Statistical analysis was assessed using the two-way ANOVA test.

FIG. 21 shows the spleen weight-to-body weight ratio of control, colitis, Bud sus, MSN-IN and Bud@MSN-IN NPs treated groups measured after animals were euthanized. Data are presented as mean±SD (n=3). *P<0.05, **P<0.01 versus DSS-induced colitis group. Statistical analysis was assessed using the one-way ANOVA test.

FIG. 22 depicts representative images and colonic damage scores of control, colitis, Bud sus, MSN-IN and Bud@MSN-IN NPs treated groups measured after animals were euthanized. Data are presented as mean±SD (n=3). *P<0.05, **P<0.01, ***P<0.001, ****P<0.0001 versus DSS-induced colitis group; ^{#}P<0.05, ^{##}P<0.01, ^{###}P<0.001, ^{####}P<0.0001 versus Bud@MSN-IN group. Statistical analysis was assessed using the one-way ANOVA test.

FIGS. 23A-23E depict representative fluorescence images showing the intracellular generation of ROS in HT-29 cells after 24 h stimulation with the following different materials in presence or absence of 200 μM H₂O₂: (FIG. 23A) Control; (FIG. 23B) H₂O₂; (FIG. 23C) H₂O₂+Bud+MSN (equivalent mass of Bud and MSN as in Bud@MSN-IN); (FIG. 23D) H₂O₂+MSN-IN (equivalent mass of MSN and inulin as in Bud@MSN-IN); (FIG. 23E) H₂O₂+Bud@MSN-IN NPs (500 μg/mL). Without treatment group was a control group. DCFH-DA was used as an intracellular ROS probe. (Scale bars, 50 μm).

FIGS. 24A-24F depict representative fluorescence images of HT-29 cells stained by calcein-AM/PI after 24 h stimulation with the following different materials in presence or absence of 200 μM H₂O₂: (FIG. 24A) Control; (FIG. 24B) H₂O₂; (FIG. 24C) H₂O₂+Inulin (equivalent mass of inulin as in Bud@MSN-IN); (FIG. 24D) H₂O₂+Bud+MSN (equivalent mass of Bud and MSN as in Bud@MSN-IN); (FIG. 24E) H₂O₂+MSN-IN (equivalent mass of MSN and inulin as in Bud@MSN-IN); (FIG. 24F) H₂O₂+Bud@MSN-IN NPs (500 μg/mL). Without treatment group was a control group. Viable cells were stained green with calcein-AM, while dead/apoptotic cells were stained red with propidium iodide (PI) (Scale bars, 50 μm).

FIG. 25 shows the changes of phylum level in fecal microbiota composition.

FIG. 26 depicts the relative abundance of Lachnospiraceae at different groups in genus level. Data are presented as mean±SD (n=4). Data was analyzed by one-way ANOVA test.

FIG. 27 depicts the relative abundance of Alloprevotella at different groups in genus level. Data are presented as mean±SD (n=4). Data was analyzed by one-way ANOVA test.

FIG. 28 depicts the relative abundance of Akkermansia at different groups in genus level. Data are presented as mean±SD (n=4). Data was analyzed by one-way ANOVA test.

FIG. 29 depicts the relative abundance of Prevotellaceae at different groups in genus level. Data are presented as mean±SD (n=4). Data was analyzed by one-way ANOVA test.

FIG. 30 shows the biodistribution of silica at 1 day, 2 days and 3 days post administration of Bud@MSN-IN NPs in healthy mice. Data are presented as mean #SD (n=3).

### DETAILED DESCRIPTION

Traditional drug delivery approaches for IBD treatment have used pH-, time-, microflora-, and pressure-triggered systems to control drug release in the colon.[39][39] Amongst the aforementioned systems, microbiota-triggered delivery systems have been found to be the most effective ones, as they exert their therapeutic effects in the presence of bacteria-derived enzymes in the colon (β-glucosidase, β-xylosidase, β-arabinosidase, β-galactosidase, nitroreductase, azoreductase, deaminase and inulinase, etc.) to further breakdown polysaccharides and dietary fibers undigested by the small intestine.[40][40] The azoreductase activity of colonic bacteria has been extensively studied for colon-targeting systems, especially in prodrug development and the Food and Drug Administration (FDA) has approved several of them for clinical use.[41][41] Notably, although the success of colon microbiota-triggered prodrug research in the evaluation of colon-targeting drug delivery, their use in clinical studies has been limited in the case of gut microbiota dysbiosis, where abundant pro-inflammatory microorganisms are considered to contribute to intestinal inflammation. However, no report has been developed so far for precise effective therapy of IBD through the colon-targeted drug release, along with effective modulation of beneficial intestinal microbiota.

The work provided herein demonstrated the feasibility and availability of orally administered Bud@MSN-IN NPs to modulate intestinal barriers, microbiome, and inflammation factors. Bud@MSN-IN NPs selectively accumulated in inflamed colon tissue and restored intestinal barrier functions by upregulating the expression levels of tight junction-associated proteins in the colon. Additionally, Bud@MSN-IN NPs treatment had a potent immune-modulatory impact in the lamina propria, characterized by significant decrease in pro-inflammatory cytokines. Moreover, Bud@MSN-IN NPs inhibited colon and mucosa damage, decreased colonic MPO activity and subsequently promoted rapid recovery from body weight loss in the DSS murine model of acute colitis. In view of the crucial role of gut microbiota dysbiosis in the etiology of IBD, [32][32] it was also found that Bud@MSN-IN NPs could reprogram the balance of gut microbiome by increasing pathogenic bacteria and reducing beneficial bacteria to alleviate and cure IBD. Thus, the observable anti-inflammatory effects of MSN-IN NPs in colitis mice may be mediated in part by changes in the gut microbiome.

In summary, this disclosure offers a precise oral strategy for colon-targeting therapy to attenuate IBD based on inulin-mediated nanoparticles. These prebiotic nanoparticles showed high stability and biocompatibility in the upper GIT and then release payload drugs upon exposure to inulinase which is presented solely in the colon, thereby improving the drug delivery efficiency and maximizing its anti-inflammatory function. Furthermore, inulin-mediated intestinal flora regulation effectively promoted inflammation elimination. Thus, the disclosure highlights the potential perspectives for prebiotic-based colon targeting therapy in IBD and other colon diseases.

The present disclosure provides, in part, a colon-targeted delivery system, composition comprising thereof, methods of making thereof, and methods of using thereof.

### Colon-Targeted Delivery Systems

Described herein are colon-targeted delivery systems that comprise mesoporous silica nanoparticles (MSN) with inulin conjugated to the surface of the MSN. The colon-targeted delivery system disclosed herein can be loaded with at least one bioactive agent, e.g., as described herein.

### Mesoporous Silica Nanoparticles

The MSN described herein are made from mesoporous materials, which are generally defined as porous inorganic solids with a pore diameter ranging from about 2 nm to about 50 nm. Provided herein are nanoparticles comprised of mesoporous silica material, referred to as mesoporous silica nanoparticles (MSNs). Methods of synthesizing MSNs are described herein.

The particle size of an MSN disclosed herein can range, e.g., from about 50 nm to about 500 nm, about 100 nm to about 500 nm, or about 200 nm to about 500 nm as measured by light-scattering methods (e.g., dynamic light scattering (DLS)), microscopy (e.g., transmission electronic microscopy (TEM)), or other appropriate methods. Particle size, as understood herein, is defined as the hydrodynamic diameter, which is the diameter of a hypothetical sphere that diffuses at the same rate as the actual particle in the same fluid. In certain embodiments, the particle size of MSNs disclosed herein is at least about or about 50 nm, 100 nm, 150 nm, 200 nm, 250 nm, 300 nm, 350 nm, 400 nm, 450 nm, or 500 nm, or any range therebetween having the listed values as endpoints. As used herein, “about” means plus or minus 10%.

The particle size distribution of MSNs disclosed herein can be uniform (monodisperse). In certain embodiments, particle size distribution of MSNs disclosed herein can be non-uniform (polydisperse). Commonly assessed by dynamic light scattering (DLS), a particle size distribution indicates the percentage of particles of a certain size. The National Institute of Standards and Technology (NIST) considers a particle distribution to be “monodisperse” if at least 90% of the distribution lies within 5% of the median size (Particle Size Characterization, Special Publication 960-1, January 2001).

Preferably, the MSNs disclosed herein can have a charged surface (i.e., zeta potential). The zeta potential refers to the measurement of the electrostatic potential at the electrical double layer surrounding a nanoparticle in solution. Zeta potential is measured in millivolts (mV) where, generally, nanoparticles with a zeta potential between −10 and +10 mV are considered approximately neutral, while nanoparticles with zeta potentials of greater than +30 mV or less than −30 mV are considered strongly cationic and strongly anionic, respectively. Zeta potential can be measured using the combination of the measurement techniques—electrophoresis and laser doppler velocimetry—also referred to as laser doppler electrophoresis. In certain embodiments, MSNs disclosed herein can have a zeta potential that ranges from about −40 mV to about −20 mV. In certain embodiments, the zeta potential of an MSN disclosed herein is at least about or about −40 mV, −35 mV, −30 mV, −25 mV, or −20 mV, or any range therebetween having the listed values as endpoints.

The specific surface area of nanoparticles (e.g., MSNs) can be measured and quantified by the Brunauer-Emmett-Teller (BET) analysis using physical gas adsorption of nitrogen gas to generate N₂ adsorption-desorption isotherms. The specific surface area obtained by this analysis is often referred to as “BET surface area” and is expressed in m²/g. In certain embodiments, MSNs disclosed herein can have a BET surface area that ranges from about 900 m²/g to about 950 m²/g. In certain embodiments, the BET surface area of an MSN disclosed herein is at least about or about 900 m²/g, 920 m²/g, 930 m²/g, 940 m²/g, or 950 m²/g, or any range therebetween having the listed values as endpoints. In certain embodiments, the BET surface area of an MSN disclosed herein is about 925 m²/g, 926 m²/g, 927 m²/g, 928 m²/g, 929 m²/g, 930 m²/g, 931 m²/g, 932 m²/g, 933 m²/g, 934 m²/g, or 935 m²/g.

The MSNs disclosed herein comprise at least one pore, and preferably comprise more than one pore. For example, the pores can be distributed throughout the entire outer surface and/or inner surface of an MSN disclosed herein, e.g., with a uniform or non-uniform distribution of the pores.

To determine total pore volume, nanoparticles (e.g., MSNs) can be subjected to the Barrett, Joyner, and Halenda (BJH) method, which complements BET by calculating pore size distributions from the same adsorption-desorption isotherms. The BJH method uses the Kelvin model of pore filling and inversely links relative pressures to pore sizes. In certain embodiments, MSNs disclosed herein can have a pore volume that ranges from about 0.3 cm³/g to about 0.9 cm³/g. In certain embodiments, the pore volume of an MSN disclosed herein is at least about or about 0.3 cm³/g, 0.4 cm³/g, 0.5 cm³/g, 0.6 cm³/g, 0.7 cm³/g, 0.8 cm³/g or 0.9 cm³/g, or any range therebetween having the listed values as endpoints.

MSNs are advantageous for the compositions (e.g., colon-targeted delivery systems) and the methods disclosed herein, in part, due to their tunable pore size. As such, tunable MSNs are embodied herein. Tuning the pore size of MSNs is essential for optimizing drug loading and controlled release. Different methods of synthesis and post-treatment strategies allow precise control over pore dimensions. These methods are described herein.

In addition to the specific surface area, the BET method can also be used to measure the “pore size” of a nanoparticle in nanometers (nm). The MSNs disclosed herein can have a pore size that ranges from about 2 nm to about 50 nm in diameter. In certain embodiments, MSNs disclosed herein can comprise pores with a size that ranges from about 2 nm or 5 nm, up to about 10, 12, 15, 20, 25 30, 35, 40, 45, or 50 nm in diameter, or any range therebetween having the listed values as endpoints.

### Inulin

Prebiotics are non-digestible carbohydrates that promote the growth and activity of beneficial bacteria in the gut. Inulin, a naturally occurring polysaccharide, is a prebiotic. Inulin is an oligosaccharide consisting of (2-1)-linked β-D-fructofuranosyl residues with an α-D-glucopyranosyl unit at the reducing end. Inulin is a soluble dietary fiber that reaches the colon undigested. Only once inulin reaches the colon is inulin fermented by the microflora of the colon. For this fermentation, the microflora produces a vast number of biodegradable enzymes, among them the enzyme inulinase. Inulinase is a digestive enzyme exclusive to the colon that hydrolyses the β-2,1 linkages of inulin to produce various intestinal metabolites, including short-chain fatty acids, that can improve gut health.

Inulin useful in the compositions and methods disclosed herein can be a modulated inulin. Methods of modulating inulin are described herein. In certain embodiments, a modulated inulin disclosed herein comprises at least one glycosidic bond. In certain embodiments, a modulated inulin disclosed herein is sensitive to enzymatic degradation by inulinase. In certain embodiments, a modulated inulin disclosed herein comprises at least one inulinase cleavage site.

### Bioactive Agents

In certain embodiments, MSNs disclosed herein can be loaded with at least one bioactive agent. Methods of loading bioactive agents are described herein. The bioactive agents that can be delivered by the compositions of the present disclosure (e.g., colon-targeted delivery systems) can be therapeutic, diagnostic, or prophylactic agents. A bioactive agent can also refer to any substance that, when administered in a therapeutically effective amount to a subject suspected of having or diagnosed as having a disease and/or disorder, has a therapeutic effect. A therapeutic effect includes, but it not limited to: (1) treating the disease/disorder; (2) slowing the progression of the disease/disorder; (3) causing the disease/disorder to retrogress or go into remission; (4) alleviating one or more symptoms of the disease/disorder; (5) reducing risk or delaying on-set of a disease/disorder; (6) maintaining (e.g., does not worsen) the disease/disorder; and/or (7) reducing risk or delaying recurrence of a disease/disorder.

Bioactive agents suitable for use herein can be, e.g., a small molecule (e.g., a drug), an organometallic compound, a polynucleotide, a polypeptide (e.g., a protein, a peptide, an antibody), a vaccine, or any combination thereof. A bioactive agent as used herein can be described as a single entity or compound or a combination of entities or compounds. A listing of bioactive agents and specific drugs suitable for use herein can be found in The Merck Index Online; Royal Society of Chemistry, 2025; rsc.org/merck-index (accessed Mar. 11, 2025), and the United States Pharmacopeia-47/National Formulary-47, published by the United States Pharmacopeial Convention, Inc., Rockville Md., 2024.

A bioactive agent suitable for use herein can also be an agent for use in diagnosis or screening. Diagnostic agents suitable for use herein include, but are not limited to, metals, commercially available imaging agents used in position emission tomography (PET), computer assisted tomography (CAT), x-ray fluoroscopy, and magnetic resonance imaging (MRI), as well as contrast agents.

A bioactive agent suitable for use herein can also be an organometallic compound. Organometallic compounds known in the art are suitable for use herein, non-limiting examples of which are discussed in Anilanmert, Beril. ‘Therapeutic Organometallic Compounds’. PHARMACOLOGY, InTech, 14 Mar. 2012.

A bioactive agent suitable for use herein can also be a polynucleotide. A polynucleotide suitable for use herein can be any nucleic acid, including but not limited to, RNA and DNA and hybrids thereof. The polynucleotides can be of any size and sequence and can be single- or double-stranded. In some embodiments, a polynucleotide suitable for use herein can be purified prior to use (e.g., prior loading into an MSN) and is substantially free from contaminants (e.g., the polynucleotide is more than about 50%, 75%, 95%, or 99% pure). A polynucleotide suitable for use herein can be obtained by any means known in the art. In some embodiments, a polynucleotide suitable for use herein can be chemically synthesized, obtained using recombinant techniques, or extracted from natural sources (e.g., a tissue sample, blood, a plant, a cell, bacteria). In some embodiments, a polynucleotide suitable for use herein can be modified by chemical and/or biological means known in the art. Methods for modification of polynucleotides include, but are not limited to, methylation, phosphorylation, end-capping, phosphate linkage, and the like. In certain embodiments, a polynucleotide suitable for use herein can be an oligonucleotide. For example, a polynucleotide suitable for use herein can be an antisense oligonucleotide (ASO), a decoy oligodeoxynucleotide (ODN), a small interfering RNA (siRNA) a short hairpin RNA (shRNA), an aptamer, a gapmer, a mixmer, an LNA, a PNA, or an mRNA.

In certain embodiments, a bioactive agent suitable for use herein can be a polypeptide, a peptide, and/or a protein. A “polypeptide”, “peptide”, or “protein” comprises a string of at least three amino acids linked together by peptide bonds. The terms “polypeptide”, “peptide”, and “protein” may be used herein interchangeably. Polypeptides suitable for use herein can comprise naturally occurring amino acids, non-naturally occurring amino acids (see, e.g., Castro T G et al, Biomolecules. 2023 Jun. 12; 13 (6): 981), and combinations thereof. In some embodiments, a polypeptide suitable for use herein can comprise at least one modified amino acid. For example, an amino acid as used herein can be modified by the addition of a chemical entity such as a carbohydrate group, a phosphate group, a farnesyl group, an isofarnesyl group, a fatty acid group, a linker for conjugation, functionalization, or other modification known in the art.

Polypeptides suitable for use herein can be antibodies. Non-limiting examples of antibodies suitable for use herein can include monoclonal antibodies, polyclonal antibodies, multispecific antibodies (e.g., bispecific antibodies), human antibodies, humanized antibodies, chimeric antibodies, single domain antibodies, camelised antibodies, single chain Fvs (scFv) single chain antibodies, Fab fragments, F(ab′) fragments, disulfide-linked Fvs (sdFv), intrabodies, and anti-idiotypic (anti-Id) antibodies, and epitope-binding fragments of any of the above. In certain embodiments, an antibody suitable for use herein specifically binds to an antigen associated with a disease and/or disorder. In certain embodiments, an antibody suitable for use herein specifically binds to an antigen associated with a colonic disease or disorder (e.g., tumor necrosis factor alpha (TNF-α), interleukin 12 (IL-12), interleukin 23 (IL-23), α4β7 integrin).

A bioactive agent suitable for use herein can be a small molecule (e.g., a small molecule drug). A “small molecule” can refer to small organic or inorganic molecules, either naturally occurring or artificially created (e.g., chemically synthesized), that have a molecular weight below about 3,000 Daltons.

In certain embodiments, a bioactive agent suitable for use herein can be a bioactive agent for treating a colonic disease or disorder. As used herein, “treating” refers to the administration of a therapeutically effective amount of a bioactive agent to a subject (e.g., a patient) suspected of having or has been diagnosed as having a colonic disease or disorder. Methods of diagnosing a subject having a colonic disease or disorder, types of colonic disease or disorder, and assessing severity of a colonic disease or disorder are known in the art and described herein.

A bioactive agent suitable for use herein can be a bioactive agent for treating an inflammatory bowel disease (IBD). IBD encompasses a group of chronic conditions that cause inflammation in the gastrointestinal (GI) tract. Two main types of IBD include ulcerative colitis (UC) and Crohn's disease. In certain embodiments, a bioactive agent suitable for use herein for treating an IBD can be an anti-inflammatory medicine (e.g., mesalamine, balsalazide, olsalazine). In certain embodiments, a bioactive agent suitable for use herein for treating an IBD can be an immunomodulator (e.g., azathioprine, mercaptopurine, methotrexate). In certain embodiments, a bioactive agent suitable for use herein for treating an IBD can be 5-Aminosalicylic acid (5-ASA; mesalamine/mesalazine). In certain embodiments, a bioactive agent suitable for use herein for treating an IBD can be a small molecule Janus kinase (JAK) inhibitor (e.g., tofacitinib, upadacitinib). In certain embodiments, a bioactive agent suitable for use herein for treating an IBD can be a small molecule sphingosine-1-phosphate (S1P) receptor modulator (e.g., ozanimod). In certain embodiments, a bioactive agent suitable for use herein for treating an IBD can be a polypeptide (also known in the art as a “biologic”). Non-limiting examples include infliximab (Remicade®), adalimumab (Humira®), golimumab (Simponi®), guselkumab (Tremfya®), certolizumab (Cimzia®), vedolizumab (Entyvio®), ustekinumab (Stelara®) and risankizumab (Skyrizi®). In certain embodiments, a bioactive agent suitable for use herein for treating an IBD can be an antibiotic (e.g., ciprofloxacin, metronidazole). In certain embodiments, a bioactive agent suitable for use herein for treating an IBD can be an oral corticosteroid (e.g., prednisone, prednisolone, budesonide). In certain embodiments, a bioactive agent suitable for use herein is budesonide.

A bioactive agent suitable for use herein can be a bioactive agent for treating a colorectal cancer. In certain embodiments, a bioactive agent suitable for use herein can be a chemotherapy drug. In some embodiments, chemotherapy drug for use herein can be an alkylating agent. Non-limiting examples of alkylating agents include altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, and trabectedin. In some embodiments, chemotherapy drug for use herein can be an antimetabolite. Non-limiting examples of antimetabolites include azacitidine, 5-fluorouracil, (5-FU), 6-mercaptopurine, (6-MP), capecitabine, (Xeloda), cladribine, clofarabine, cytarabine, (Ara-C), decitabine, floxuridine, fludarabine, gemcitabine, (Gemzar), hydroxyurea, methotrexate, nelarabine, pemetrexed, (Alimta), pentostatin, pralatrexate, thioguanine, and a trifluridine/tipiracil combination. In some embodiments, chemotherapy drug for use herein can be an anthracycline. Non-limiting examples of anthracyclines include daunorubicin, doxorubicin (adriamycin), doxorubicin liposomal, epirubicin, idarubicin, and valrubicin. In some embodiments, chemotherapy drug for use herein can be a topoisomerase inhibitor. Non-limiting examples of topoisomerase inhibitors include irinotecan, irinotecan liposomal, topotecan, etoposide (VP-16), mitoxantrone, and teniposide. In some embodiments, chemotherapy drug for use herein can be a mitotic inhibitor. Non-limiting examples of mitotic inhibitors include cabazitaxel, docetaxel, nab-paclitaxel, paclitaxel, vinblastine, vincristine, vincristine liposomal, and vinorelbine. In certain embodiments, a bioactive agent suitable for use herein can be a chemotherapy drug used to treat colorectal cancer. In certain embodiments, a bioactive agent suitable for use herein can be 5-Fluorouracil (5-FU), capecitabine, irinotecan hydrochloride, folinic acid (also known as leucovorin, leucovorin calcium, or calcium folinate), oxaliplatin, trifluridine and tipiracil hydrochloride (Lonsurf®), or any combination thereof.

### Preparation of Colon-Targeted Delivery Systems

A colon-targeted delivery system can be prepared using at least one MSN and at least one inulin. Preferably, a colon-targeted delivery system can be prepared using at least one MSN, at least one inulin, and at least one bioactive agent.

### Synthesis and Functionalization of MSNs

Methods of making MSNs are known in the art (see, e.g., Pande V et al., “Fabrication of Mesoporous Silica Nanoparticles and Its Applications in Drug Delivery” NANOTECHNOLOGY AND NANOMATERIALS. IntechOpen; 2023). Non-limiting examples of methods for synthesizing MSNs for use herein include template-assisted synthesis, sol-gel methods, and co-condensation methods. In certain embodiments, MSNs of the present disclosure can be synthesized using a sol-gel reaction (see, e.g., Pande V et al., “Fabrication of Mesoporous Silica Nanoparticles and Its Applications in Drug Delivery” NANOTECHNOLOGY AND NANOMATERIALS. IntechOpen; 2023).

Tuning the pore size of MSNs is advantageous for the compositions and the methods disclosed herein, in part, for optimizing loading of a bioactive agent and its controlled release. By selecting the appropriate method of synthesis, MSNs disclosed herein can be tuned for encapsulating different types of therapeutic agents, ensuring precise, controlled release of bioactive agent(s).

MSNs with pores with sizes that range from about 2 nm to about 5 nm can be generated, e.g., by adjusting cetyltrimethylammonium bromide (CTAB) concentration during MSN synthesis, similar to the methods described in Carvalho G C et al., Adv Colloid Interface Sci. 2022 September; 307:102746.

MSN with pores with sizes that range from about 5 nm to about 10 nm can be generated, e.g., by using polymer-assisted methods (P123, F127) to form larger micelles during MSN synthesis, similar to the methods described in Guha A et al., Curr Drug Deliv. 2016; 13 (4): 574-81.

MSNs with pores with a size that ranges from about 5 nm to about 12 nm in diameter can be generated, e.g., by alkaline etching, similar to the methods described in Abdollahi L et al. Biomater Adv. 2022 February; 133:112621, where controlled dissolution enlarges existing pores.

MSNs with pores with a size that ranges up to about 15 nm in diameter can be generated, e.g., by the addition of swelling agents like TMB, mesitylene, and xylene, which expand micelle size during MSN synthesis, similar to the methods described in Matos J R et al., J Am Chem Soc. 2003 Jan. 22; 125 (3): 821-9.

MSNs with pore sizes larger than 15 nm can be synthesized using a biphasic stratification approach, as described by Noureddine et al., ACS Nano. 2023 Sep. 12; 17 (17): 16308-16325. In this method, a dendritic structure with large pores is formed at the interface between an aqueous surfactant-rich phase and an organic phase containing the silica precursor (e.g., tetraethyl orthosilicate, TEOS). By carefully adjusting the concentration of TEOS in the organic phase, pore sizes can be reliably tuned from approximately 4 nm to over 15 nm, reaching up to about 25 nm or up to about 50 nm. Controlling TEOS concentration and reaction conditions during synthesis allow for the formation of large and highly accessible dendritic pores (see, e.g., Noureddine et al., ACS Nano. 2023 Sep. 12; 17 (17): 16308-16325).

In certain embodiments, MSNs disclosed herein can be functionalized. As understood herein, “functionalizing” the MSNs of the present disclosure involves attaching at least one organic and/or inorganic group to their surface. There are many methods known in the art that can be used to functionalize MSNs. However, most of these methods are not suitable for preparing the MSN-inulin conjugate provided by the present disclosure. Basic methods such as electrostatic adsorption and covalent bonding via salinization are not suitable methods for attaching inulin to MSN to arrive at the MSN-inulin conjugate contemplated herein because these methods require inulin to be modified in a manner that prevents its cleavage by the colon-specific inulinase. As such, the present disclosure provides amination of MSNs in order to functionalize the MSN. In certain embodiments, an MSN disclosed herein can comprise at least one amine group on its surface. In certain embodiments, an MSN disclosed herein is functionalized by amination (e.g., MSN-NH₂).

### Synthesis and Modification of Inulin

Methods of synthesizing inulin are known in the art. Generally, inulin is typically synthesized through an enzymatic process using sucrose as the starting material, where an enzyme (e.g., fructansucrase) progressively adds fructose units to the sucrose molecule, creating a chain of fructose linked by beta (2-1) glycosidic bonds, essentially building the inulin polymer, e.g., as described in Ni et al., Biotechnol Adv. 2019 March-April; 37 (2): 306-318. The molecular weight of inulin can depend on the chain length of the fructose units added during synthesis. In certain embodiments, the molecular weight of inulin can range from about 3,500 to about 5,500 Daltons (Da).

In certain embodiments, inulin for use in the compositions disclosed herein (e.g., colon-targeted delivery systems) can be modified. As described above, it is a modified inulin for use herein preferably comprises certain properties (e.g., comprising at least one glycosidic bond, comprising at least one inulinase cleavage site). In certain embodiments, a modulated inulin can be carboxymethyl inulin (CMI). CMI can be synthesized by carboxymethylation of the carboxyl side chains on inulin by reacting inulin with sodium salt of monochloro acetic acid (SMCA) in presence of sodium hydroxide (see, e.g., U.S. Pat. No. 7,728,124).

### Loading of Bioactive Agents

The present disclosure provides colon-targeted delivery systems for delivering one or more bioactive agents to the colon. Once loaded into the MSN disclosed herein, the bioactive agent can also be referred to as the “payload” or “cargo.” As discussed herein, the MSNs of the present disclosure are tunable, which allows for a range of bioactive agents to be loaded into the MSNs. Once the bioactive agent to be delivered to the colon is decided upon, MSNs with a suitable pore size can be synthesized according to the appropriate methods described above. In certain embodiments, at least one bioactive agent can be loaded into an MSN herein. In certain embodiments, at least one bioactive agent can be loaded into a functionalized MSN (e.g., an MSN-NH₂) disclosed herein.

Methods of loading bioactive agents into MSNs are generally known in the art can vary depending on the type of bioactive agent to be loaded and/or pore size of the MSN. In certain embodiments, the bioactive agent to be loaded into an MSN/functionalized MSN disclosed herein is a small molecule (e.g., a molecule comprising a molecular weight ranging from about 100 Da to about 600 Da) and the MSN/functionalized MSN comprises pores ranging from about 2 nm to about 5 nm in size. In certain embodiments, a small molecule can be loaded into an MSN/functionalized MSN by electrostatic interaction or hydrogen bonding (see, e.g., Manzano, M et al., (2009) Expert Opinion on Drug Delivery, 6 (12), 1383-1400; and Vallet-Regí, M et al., (2001) Chemistry of Materials, 13 (2), 308-311).

In certain embodiments, the bioactive agent to be loaded into an MSN/functionalized MSN disclosed herein is a polypeptide (e.g., a polypeptide comprising a molecular weight ranging from about 4 kDa to about 15 kDa) and the MSN/functionalized MSN comprises pores ranging from about 5 nm to about 10 nm in size. In certain embodiments, a polypeptide can be loaded into an MSN/functionalized MSN by nanoprecipitation or an emulsion-based technique (see, e.g., Bilati, U et al., (2005) AAPS PharmSciTech 6, 74; Tang, F., et al. (2012). Advanced Materials, 24 (12), 1504-1534; and Huang, Y., et al. (2022). Pharmaceutics, 15 (1), 89).

In certain embodiments, the bioactive agent to be loaded into an MSN/functionalized MSN disclosed herein is a polynucleotide and the MSN/functionalized MSN comprises pores ranging from about 10 nm to about 15 nm in size. In certain embodiments, the bioactive agent is a polynucleotide and the polynucleotide can be surface-attached to an MSN/functionalized MSN disclosed herein. Methods for loading polynucleotides into MSNs and attaching polynucleotides to an MSN surface are known in the art (see, e.g., Xie, X., et al. (2023). Pharmaceutics, 15 (10), 2483), and are suitable for use herein.

In certain embodiments, the bioactive agent is a hydrophobic drug (e.g., a hydrophobic drug comprising a molecular weight ranging from about 800 Da to about 1000 Da) and is encapsulated in an MSN/functionalized MSN disclosed herein. In certain embodiments, a hydrophobic drug is encapsulated in an MSN/functionalized MSN that comprises at least one hydrophobic surface modification. Methods for loading hydrophobic drug into MSNs that comprise at least one hydrophobic surface modification are known in the art (see, e.g., Wu, S. H., et al. (2013). Chemical Society Reviews, 42 (9), 3862-3875), and are suitable for use herein.

In certain embodiments, at least two bioactive agents are loaded into an MSN/functionalized MSN disclosed herein. Methods of loading more than one bioactive agent into MSN are known in the art (see, e.g., Galhano, J., et al. (2022). Bioorganic Chemistry, 118, 105470), and are suitable for use herein.

In certain embodiments, methods of loading at least one bioactive agent (also referred to herein as the payload or cargo) have a loading efficiency of at least about 20%. In certain embodiments, methods of loading the payload have a loading efficiency that exceeds about 20%. In certain amendments, methods of loading the payload have a loading efficiency ranging from about 20% to about 100%.

### Synthesis of MSN-Inulin Conjugates

The present disclosure provides a colon-targeted delivery system comprising at least one MSN-inulin (MSN-IN) conjugate. In certain embodiments, inulin is conjugated to the surface of an MSN disclosed herein. In certain embodiments, a modified inulin (e.g., CMI) is conjugated to the surface of an MSN disclosed herein. In certain embodiments, a modified inulin (e.g., CMI) is conjugated to the surface of a functionalized MSN (e.g., an MSN-NH₂) disclosed herein. In certain embodiments, inulin/modified inulin is conjugated to the surface of an MSN, wherein the MSN has been previously loaded with at least one bioactive agent as described above.

In certain embodiments, inulin is covalently conjugated to the surface of an MSN disclosed herein. As previously described, the method of conjugating inulin to MSN should preserve the inulinase cleavage site. As a result, these MSNs conjugated with inulin undergo controlled degradation in response to inulinase, making them effective for triggered drug release specifically in the colon. In certain embodiments, a modified inulin is covalently conjugated to the surface of a functionalized MSN. In certain embodiments, a CMI is covalently conjugated to the surface of an aminated MSN (e.g., MSN-NH₂). In certain embodiments, a modified inulin is covalently conjugated to the surface of a functionalized MSN. In certain embodiments, a CMI is covalently conjugated to the surface of an aminated MSN (e.g., MSN-NH₂) by EDC/NHS chemistry. EDC/NHS chemistry is a reaction that uses the compounds 1-ethyl-3-(3-dimethylaminopropyl) carbodiimide (EDC) and N-hydroxysuccinimide (NHS) to link amine-containing biomolecules (e.g., MSN-NH₂) to carboxyl-containing substrates (e.g., CMI). Conjugation by EDC/NHS chemistry is generally known in the art (see, e.g., Fischer M J, Methods Mol Biol. 2010; 627:55-73) and can be used and/or modified for use herein.

### Characteristics of the Colon-Targeted Delivery Systems

The colon-targeted delivery systems provided by the present disclosure are enzyme-activated drug delivery systems designed for oral administration with targeted colon delivery. The colon-targeted delivery systems of the present disclosure are prepared using the materials and methods described in the sections above to utilize inulinase, an enzyme exclusively present in the colon, to trigger therapeutic payload release into the colon. Unlike conventional methods that rely on pH sensitivity or time-dependence, this approach ensures precise activation in the colon while protecting the drug from degradation in the stomach and upper GI tract.

In certain embodiments, a colon-targeted delivery system of the present disclosure comprises an MSN, inulin conjugated to the surface of the MSN, and at least one bioactive agent loaded into the MSN. In certain embodiments, a colon-targeted delivery system of the present disclosure comprises an MSN and inulin conjugated to the surface of the MSN, wherein the addition of a least one bioactive agent loaded into the MSN is optional.

In certain embodiments, the particle size of a colon-targeted delivery system (e.g., a composition) disclosed herein, can range from about 50 nm to about 500 nm, about 100 nm to about 500 nm, or about 200 nm to about 500 nm. In certain embodiments, the particle size of a composition disclosed herein is at least about or about 50 nm, 100 nm, 150 nm, 200 nm, 250 nm, 300 nm, 350 nm, 400 nm, 450 nm, 500 nm, or any range therebetween having the listed values as endpoints. In certain embodiments, the particle size of a composition disclosed herein is about 270 nm, 271 nm, 272 nm, 273 nm, 274 nm, 275 nm, 276 nm, 277 nm, 278 nm, 279 nm, or 280 nm. In certain embodiments, the particle size of a composition disclosed herein is about 275 nm.

In certain embodiments, particle size distribution of colon-targeted delivery systems disclosed herein can be uniform (monodisperse). In certain embodiments, particle size distribution of colon-targeted delivery systems disclosed herein can be non-uniform (polydisperse).

In certain embodiments, a colon-targeted delivery system (e.g., a composition) disclosed herein can have a charged surface (i.e., zeta potential). In certain embodiments, a composition disclosed herein can have a zeta potential that ranges from about −20 mV to about −10 mV. In certain embodiments, the zeta potential of a composition disclosed herein is at least about or about −20 mV, −15 mV, −10 mV, or any range therebetween having the listed values as endpoints. In certain embodiments, the zeta potential of a composition disclosed herein is about −14 mV, −15 mV, −16 mV, −17 mV, −18 mV, or −19 mV.

In certain embodiments, a colon-targeted delivery system (e.g., a composition) disclosed herein can have a BET surface area that ranges from about 20 m²/g to about 100 m²/g. In certain embodiments, the BET surface area of a composition disclosed herein is at least about or about 20 m²/g, 40 m²/g, 60 m²/g, 80 m²/g, 100 m²/g, or any range therebetween having the listed values as endpoints. In certain embodiments, the BET surface area of a composition disclosed herein is about 34 m²/g, 35 m²/g, 36 m²/g, 37 m²/g, 38 m²/g, 39 m²/g, 40 m²/g, 41 m²/g, 42 m²/g, 43 m²/g, or 44 m²/g.

In certain embodiments, colon-targeted delivery systems disclosed herein can comprise at least one pore. In certain embodiments, colon-targeted delivery systems disclosed herein can comprise more than one pore. In certain embodiments, the pores can be throughout the entire outer surface and/or inner surface of a composition disclosed herein. In certain embodiments, a composition disclosed herein can comprise a uniform distribution of more than one pore.

In certain embodiments, a colon-targeted delivery system (e.g., a composition) disclosed herein can have a pore volume that ranges from about 0.02 cm³/g to about 0.08 cm³/g. In certain embodiments, the pore volume of a composition disclosed herein is at least about or about 0.02 cm³/g, 0.03 cm³/g, 0.04 cm³/g, 0.05 cm³/g, 0.06 cm³/g, 0.07 cm³/g, 0.08 cm³/g, or any range therebetween having the listed values as endpoints. In certain embodiments, the pore volume of a composition disclosed herein is about 0.03 cm³/g, 0.04 cm³/g, or 0.05 cm³/g. In certain embodiments, a composition disclosed herein can have a pore volume that ranges from about 2 nm to about 25 nm in diameter.

Colon-targeted delivery systems provided herein protect the encapsulated payload (e.g., bioactive agent) during gastric transit. In certain embodiments, a colon-targeted delivery system (e.g., a composition) of the present disclosure releases less than about 10% of the payload in the upper GI tract. In certain embodiments, a composition of the present disclosure releases less than about 10% of the payload in an environment having a pH ranging from 1 to 7. In certain embodiments, a composition of the present disclosure releases less than about 10% of the payload in gastric fluid. In certain embodiments, a composition of the present disclosure releases less than about 10% of the payload in intestinal fluid. In certain embodiments, in the absence of inulinase, a composition of the present disclosure releases less than about 10% of the payload.

In certain embodiments, a colon-targeted delivery system (e.g., a composition) of the present disclosure releases at least about 60% of the payload (e.g., bioactive agent) in the lower GI tract. In certain embodiments, a composition of the present disclosure releases more than about 60% of the payload in the lower GI tract. In certain embodiments, a composition of the present disclosure releases at least about 60% of the payload in the colon. In certain embodiments, a composition of the present disclosure releases more than about 60% of the payload in the colon. In certain embodiments, a composition of the present disclosure releases more than about 60% of the payload in an environment having a pH above 7. In certain embodiments, a composition of the present disclosure releases more than about 60% of the payload in colon fluid. In certain embodiments, in the presence of inulinase, a composition of the present disclosure releases more than about 60% of the payload.

In certain embodiments, a colon-targeted delivery system (e.g., a composition) of the present disclosure releases the payload (e.g., bioactive agent) instantly in the colon (e.g., in the presence of inulinase). In certain embodiments, a composition of the present disclosure releases the payload over time in the colon. In certain embodiments, a composition of the present disclosure releases the payload over a frame of time ranging from about 5 minutes to about 48 hours, about 30 minutes to about 36 hours, or about 1 hour to about 24 hours in the colon. In certain embodiments, a composition of the present disclosure releases more than about 60% of the payload in the colon over a frame of time ranging from about 5 minutes to about 48 hours, about 30 minutes to about 36 hours, or about 1 hour to about 24 hours in the colon. In certain embodiments, a composition of the present disclosure releases more than about 60% of the payload in the colon over at least about or about 5 minutes, 10 minutes, 15 minutes, 30 minutes, 45 minutes, 1 hour, 1.5 hours, 2 hours, 3 hours, 4 hours, 5 hours, 6 hours, 12 hours, 24 hours, 36 hours, or 48 hours. In certain embodiments, a composition of the present disclosure releases about 60% to about 100% of the payload in the colon over at least about or about 5 minutes, 10 minutes, 15 minutes, 30 minutes, 45 minutes, 1 hour, 1.5 hours, 2 hours, 3 hours, 4 hours, 5 hours, 6 hours, 12 hours, 24 hours, 36 hours, or 48 hours.

### Pharmaceutical Compositions and Methods of Administration

The methods described herein include the use of pharmaceutical compositions comprising or consisting of at least one colon-targeted delivery system as described herein.

Pharmaceutical compositions typically include a pharmaceutically acceptable carrier. As used herein, the language “pharmaceutically acceptable carrier” includes saline, solvents, dispersion media, coatings, antibacterial and antifungal agents, isotonic and absorption delaying agents, and the like, compatible with pharmaceutical administration. Acceptable carriers, excipients, or stabilizers are nontoxic to recipients at the dosages and concentrations used, and comprise buffers such as phosphate, citrate, and other organic acids; antioxidants including ascorbic acid and methionine; preservatives (such as octadecyl dimethyl benzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, di saccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbate, pluronic polymers, or polyethylene glycol (PEG). In certain embodiments, a pharmaceutical composition of the present disclosure comprises at least one colon-targeted delivery system and at least one pharmaceutically acceptable carrier.

In certain embodiments, pharmaceutical compositions disclosed herein can also contain more than one bioactive agent (e.g., a supplemental bioactive agent) as necessary, preferably those with complementary activities that do not adversely affect each other. In certain embodiments, a supplemental bioactive agent included in a pharmaceutical composition disclosed herein can comprise any one of the bioactive agents for treating a colonic disease or disorder and/or a symptom thereof as described herein.

Pharmaceutical compositions are typically formulated to be compatible with their intended route of administration. In certain embodiments, a pharmaceutical composition of the present disclosure comprises at least one colon-targeted delivery system and at least one pharmaceutically acceptable carrier formulated for oral administration.

In certain embodiments, pharmaceutical compositions described herein can be in unit dosage forms such as tablets, pills, capsules, powders, granules, solutions or suspensions, for oral administration. For preparing solid pharmaceutical compositions such as tablets, the principal active ingredient (e.g., at least one colon-targeted delivery system) can be mixed with a pharmaceutical carrier, e.g., conventional tableting ingredients such as com starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of the principal active ingredient, or a non-toxic pharmaceutically acceptable salt thereof. When referring to these preformulation compositions as homogeneous, it is meant that the principal active ingredient is dispersed evenly throughout the composition so that the composition can be readily subdivided into equally effective unit dosage forms such as tablets, pills and capsules.

### Kits

In certain embodiments, the present disclosure provides kits for use in treating, preventing, or alleviating a colonic disease or disorder in a subject described herein. For example, the kits can include, but are not limited to, one or more containers including, for example, pharmaceutical compositions comprising the colon-targeted delivery system disclosed herein.

In certain embodiments, kits disclosed herein can include instructions for use in accordance with any of the methods described herein. The included instructions can have a description of administration of the pharmaceutical compositions to treat, delay the onset, or alleviate a target disease such as those described herein.

In certain embodiments, kits disclosed herein can further include a description of selecting an individual suitable for treatment based on identifying whether that individual has the target disease, e.g., applying the diagnostic methods as described herein. In certain embodiments, the instructions can have a description of administering any one of the compositions described herein to an individual at risk of the target disease.

The kits can include instructions relating to the use of the pharmaceutical compositions described herein, e.g., information as to dosage, dosing schedule, and route of administration for the intended treatment. The containers can be unit doses, bulk packages (e.g., multi-dose packages) or sub-unit doses. Instructions supplied in the kits disclosed herein are typically written instructions on a label or package insert (e.g., a paper sheet included in the kit), but machine-readable instructions (e.g., instructions carried on a magnetic or optical storage disk) are also acceptable. The label or package insert can indicate that the pharmaceutical composition is used for treating, delaying the onset and/or alleviating the target disease (e.g., a colonic disease or disorder).

### Methods of Use

Provided herein are methods of administering a colon-targeted delivery system or a pharmaceutical composition comprising thereof to a subject in need thereof. In certain embodiments, a method of administering a colon-targeted delivery system or a pharmaceutical composition comprising thereof can be used to treat, prevent (reduce risk of), and/or ameliorate a colonic disease or disorder. In certain embodiments, a method of administering a colon-targeted delivery system or a pharmaceutical composition comprising thereof can be used to treat, prevent, and/or ameliorate at least one symptom of a colonic disease or disorder.

### Subject Populations

Methods of administering a colon-targeted delivery system or a pharmaceutical composition, as provided in the present disclosure, are suitable for use in a subject in need thereof. “Subject” and “patient” refer to either a human or non-human, such as mammals, e.g., vertebrates, e.g., primates, that express inulinase in their colon. In certain embodiments, the subject is a human or a non-human veterinary subject such as a non-human primate, cat, dog, horse, cow, goat, or rabbit.

In certain embodiments, a subject in need thereof is a subject suspected of having or has been diagnosed as having a colonic disease or disorder. Colonic diseases and disorders refer to a group of conditions that affect the colon (e.g., the large intestine). Colonic diseases and disorders can cause a wide range of symptoms, including, but not limited to, abdominal pain, diarrhea, constipation, bloating, rectal bleeding, and fever. Methods of diagnosing a subject with a colonic disease and/or disorder are known in art and include, but are not limited to, blood tests, stool tests, colonoscopy, sigmoidoscopy, and/or medical imaging (e.g., CT, MRI, ultrasound, etc). In certain embodiments, a subject in need thereof is a subject suspected of having or has been diagnosed as having a colonic disease or disorder selected from the group consisting of colorectal cancer, colonic polyps, Crohn's disease, diverticulitis, inflammatory bowel disease (IBD), irritable bowel syndrome (IBS), microscopic colitis, and ulcerative colitis (UC).

In certain embodiments, a subject in need thereof is a subject suspected of having or has been diagnosed as having IBD. IBD refers to a group of disorders that cause the intestines to become inflamed, generally manifested with symptoms including abdominal cramps and pain, diarrhea, weight loss and intestinal bleeding. The most common types of IBD are ulcerative colitis (UC) and Crohn's disease.

In certain embodiments, a subject in need thereof is a subject suspected of having or has been diagnosed as having UC. UC is a chronic, episodic, inflammatory disease of the large intestine and rectum characterized by bloody diarrhea. UC is characterized by chronic inflammation in the colonic mucosa and can be further categorized according to location: “proctitis” involves only the rectum, “proctosigmoiditis” affects the rectum and sigmoid colon, “left-sided colitis” encompasses the entire left side of the large intestine, “pancolitis” inflames the entire colon. In certain embodiments, a subject in need thereof is a subject suspected of having or has been diagnosed as having proctitis, proctosigmoiditis, left-sided colitis, pancolitis, or any combination thereof. A disease activity index (DAI) scoring system is a method for quantitatively assessing UC activity. The DAI score is a simplified composite score incorporating four variables: stool frequency, rectal bleeding, mucosal appearance, and physician's rating of disease activity (see Sutherland L R et al., Gastroenterology 1987; 92:1894-8).

In certain embodiments, a subject in need thereof is a subject suspected of having or has been diagnosed as having Crohn's disease. Crohn's disease is a chronic autoimmune disease that can affect any part of the gastrointestinal tract but most commonly occurs in the ileum (the area where the small and large intestine meets). Crohn's disease, in contrast to UC, is characterized by chronic inflammation extending through all layers of the intestinal wall and involving the mesentery as well as regional lymph nodes. UC and Crohn's disease can be distinguished from each other clinically, endoscopically, pathologically, and serologically in more than 90% of cases (see, e.g., Harrison's Principles of Internal medicine, 12^{th} edition, 1991).

For IBDs, activity can be assessed according to diagnostic measures known in the art. For example, disease severity may be assessed by a sigmoidoscopy score. Following a sigmoidoscopy, a score can be assigned as follows: 0=normal or inactive disease; 1=mild disease (erythema, decreased vascular pattern, mild friability); 2=moderate disease (marked erythema, absent vascular pattern, friability, erosions); 3=severe disease (spontaneous bleeding, ulceration). IBD severity can also be assessed by a rectal bleeding score. A rectal bleeding score is the score or grade assigned for the extent, if any, of rectal bleeding where the preferred rectal bleeding scoring system is: 0=no blood seen; 1=streaks of blood with stool less than half the time; 2=obvious blood with stool most of the time; 3=blood alone passed.

In certain embodiments, a subject in need thereof is a subject suspected of having or has been diagnosed as having a colorectal cancer. Colorectal cancer is a type of cancer that develops in the colon or rectum, (e.g., in the large intestine). In certain embodiments, a subject in need thereof is a subject suspected of having or has been diagnosed as having a metastatic tumor located in the colon. Symptoms of colorectal cancer include, but are not limited to, changes in bowel habits (e.g., diarrhea, constipation, abdominal pain), rectal bleeding or blood in the stool, weight loss, fatigue, bloating, nausea, and vomiting. Methods of diagnosing a subject with a colorectal cancer are known in the art and include, but are not limited to blood tests, colonoscopy, tissue biopsy, and/or medical imaging (e.g., CT, MRI, ultrasound, etc). Once diagnosed with colorectal cancer, a subject herein can be subjected to staging to assess the severity of the cancer. The earliest stage of colorectal cancers is stage 0 (a very early cancer), and then range from stages I (1) through IV (4) with stage IV colorectal cancer being the most advanced (see, e.g., American Joint Committee on Cancer. Chapter 20-Colon and Rectum. In: AJCC Cancer Staging Manual. 8th ed. New York, NY: Springer; 2017). In certain embodiments, a subject in need thereof is a subject diagnosed as having stage I colorectal cancer, stage II colorectal cancer, stage III colorectal cancer, or stage IV colorectal cancer.

### Methods of Treatment and Treatment Outcomes

The present disclosure provides methods of treating a subject suspected of having or has been diagnosed as having a colonic disease or disorder as disclosed herein. In certain embodiments, a therapeutically effective amount of a colon-targeted delivery system or a pharmaceutical composition as described herein can be administered to a subject in need thereof. In certain embodiments, a therapeutically effective amount of a colon-targeted delivery system or a pharmaceutical composition as described herein can be administered to a subject in need thereof in combination with at least one biological agent disclosed herein. In certain embodiments, a therapeutically effective amount of a colon-targeted delivery system or a pharmaceutical composition as described herein can be administered to a subject in need thereof in combination with at least one non-pharmacologic therapy for treating a colonic disease or disorder. Non-limiting examples of non-pharmacologic therapy for treating a colonic disease or disorder can include surgery, diet, radiation, ablation, and the like.

In certain embodiments, a therapeutically effective amount of a colon-targeted delivery system or a pharmaceutical composition can result in an improvement in at least one symptom of the target disease (e.g., a colonic disease or disorder) following administration. In certain embodiments, a therapeutically effective amount of a colon-targeted delivery system or a pharmaceutical composition can result in a reduction in a subject's DAI score following administration. In certain embodiments, a therapeutically effective amount of a colon-targeted delivery system or a pharmaceutical composition can result in a reduction in a subject's sigmoidoscopy score following administration. In certain embodiments, a therapeutically effective amount of a colon-targeted delivery system or a pharmaceutical composition can result in a reduction in a subject's rectal bleeding score following administration.

In certain embodiments, a therapeutically effective amount of a colon-targeted delivery system or a pharmaceutical composition can prevent progression colonic disease or disorder following administration. In certain embodiments, a therapeutically effective amount of a colon-targeted delivery system or a pharmaceutical composition can prevent an increase in a subject's DAI score, sigmoidoscopy score, and/or rectal bleeding score following administration. In certain embodiments, a therapeutically effective amount of a colon-targeted delivery system or a pharmaceutical composition can prevent a colorectal cancer in a subject from progressing to the next stage, from metastasizing, or both. In certain embodiments, a therapeutically effective amount of a colon-targeted delivery system or a pharmaceutical composition can lead to remission of colonic disease or disorder following administration.

In certain embodiments, a therapeutically effective amount of a colon-targeted delivery system or a pharmaceutical composition can restore or prevent progression of a pathology associated with a colonic disease or disorder following administration. In certain embodiments, a therapeutically effective amount of a colon-targeted delivery system or a pharmaceutical composition can restore the colonic epithelium from the damage resulting from a colonic disease or disorder following administration. In certain embodiments, a therapeutically effective amount of a colon-targeted delivery system or a pharmaceutical composition can prevent further damage to the colonic epithelium as a result of a colonic disease or disorder following administration.

The present disclosure also provides methods of modulating the composition of the gut microbiome in a subject in need thereof. In certain embodiments, a therapeutically effective amount of a colon-targeted delivery system or a pharmaceutical composition can modulate the composition of the gut microbiome in a subject following administration. In certain embodiments, a therapeutically effective amount of a colon-targeted delivery system or a pharmaceutical composition can increase the amount of Firmicutes, Bacteroidetes, Verrucomicrobia, Alloprevotella, Lactobacillus, Akkermansia, Prevotellaceae, and/or Proteobacteria phyla in the fecal microbiota following administration. In certain embodiments, a therapeutically effective amount of a colon-targeted delivery system or a pharmaceutical composition can increase the amount of Alloprevotella, Lactobacillus, Akkermansia, and/or Prevotellaceae in the fecal microbiota following administration. In certain embodiments, a therapeutically effective amount of a colon-targeted delivery system or a pharmaceutical composition can decrease the amount of pathogenic bacteria in the fecal microbiota following administration. In certain embodiments, a therapeutically effective amount of a colon-targeted delivery system or a pharmaceutical composition can decrease the amount of Alistipes and/or Lachnospiraceae in the fecal microbiota following administration.

### EXAMPLES

The invention is further described in the following examples, which do not limit the scope of the invention described in the claims.

### Example 1: Prebiotic Nanomedicine for Targeted Colon Therapy of Inflammatory Bowel Disease

Inflammatory bowel disease (IBD), typically sub-grouped as ulcerative colitis and Crohn's disease, is an incurable chronic inflammatory colorectal disease that afflict millions worldwide.[1][1] Current treatment strategies are mostly conservative and only rely on conventional free drugs including anti-inflammatory molecules, immunosuppressants, and other similar biological components to slow down symptom flare-ups and maintain remission.[2][2] However, these conventional drugs are frequently associated with recurrence and systemic side-effects due to their nonspecific immunomodulatory effects and long-term systemic exposure.[2b, 3][2b, 3] Moreover, these conventional drugs are typically absorbed in the upper gastrointestinal tract (GIT) before they reach the site of disease in the colon, thus making their therapeutic efficacy far from satisfactory. Although the exact causes of IBDs remain unknown, intestinal dysbiosis correlates with increased IBD incidence, suggesting that the gut microbiota play a major role in regulating disease pathogenesis.[4][4] However, the cure of IBD, associated with intestinal dysbiosis and aberrant immune response, remain elusive to date. Especially, no treatments can effectively prevent such disease progression and achieve complete cure. Thus, a novel therapeutic strategy that can target the IBD-affected lower GIT, reprogram the gut microbiome balance and alleviate intestinal inflammation without impacting the surrounding microbial ecosystem remains a daunting and unmet challenge.

In this regard, nanotherapeutics and nano-based delivery platforms provide new opportunity for IBD therapy.^{[5][5]} Colon-targeted oral drug delivery systems (CDDSs) are desirable for IBD treatment, since the major inflammation location is the colon.^{[6][6]} Currently, various colon-targeted formulation strategies have been developed under the physiological conditions of the gastrointestinal tract (GIT), including pH-sensitive polymer coatings^{[7][7]}, time-dependent release systems^{[8][8]}, and prodrug formulations^{[9][9]}. Despite these advancements, the existing nano-strategies still have certain limitations. For example, the reliability of pH-sensitive polymers for colon drug delivery has been questioned for some time.^{[10][10]} Human studies have shown that in IBD, the intestinal pH is often markedly lower than that of healthy individuals.^{[11][11]} Consequently, the disease pathophysiologypathophysiology can increase the risk of pH-sensitive polymer-based strategies failure. The main limitation of time-dependent colonic delivery strategy is that this approach can be highly affected by variability in gastrointestinal (GI) transit time. Many factors, including presence of disease, surgery, concurrent medications, or a change in diet, can alter patients' GI transit times and lead to time-based approaches failing to predictably deliver drugs to the colon. These key limitations highlight the glaring need for new biocompatible and effective colon-targeted oral administered drug delivery systems for IBD. In recent years, increasing characterization of the colonic environments has led to the recognition of new local targets, such as the microbiome, enteric immune system, and lymphatic system.^{[11-12][11-12]} These emerging targets not only allow targeted drug delivery within the colon, but also facilitate the treatment of colonic pathologies and even impact distal organs.^{[6b, 13][6b, 13]} Considering the important role of gut microbiota in the occurrence and development of IBD, the gut microbiota represents an attractive emerging therapeutic target that has been widely explored for targeted treatment of IBD.

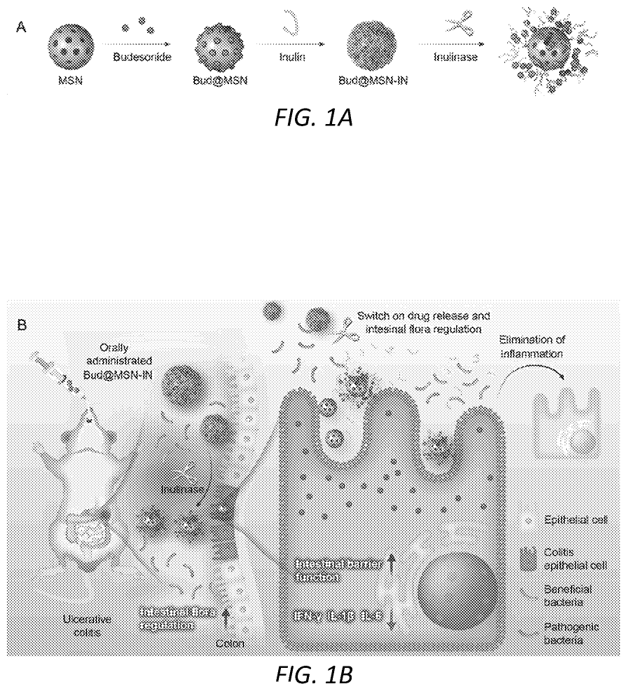

Herein, for more effective therapy of inflammatory bowel disease, orally administered inulin-mediated prebiotic nanoparticles were developed for precise colon-targeted “switch on” drug release and regulation of gut microbiota. Budesonide (Bud), an oral corticosteroid that is recommended by the American College of Gastroenterology (ACG) as the clinical “first-choice” drug for patients with mild to moderate active IBD in whom 5-ASA induction therapy fails or is not tolerated.^{[14][14]} However, due to the poor solubility and 90% hepatic first-pass metabolism of Bud, it results in low transport efficiency and low bioavailability, leading to insufficient drug concentration in inflamed colon tissue. Additionally, increased dosages exacerbate systemic side effects, making it difficult to achieve the desired therapeutic effect.^{[15][15]} To improve the bioavailability of Bud, mesoporous silica nanoparticles (MSNs) were used as drug carriers to encapsulate it within nanoparticles. In which, MSNs have been widely used as oral drug delivery carriers due to their uniform mesopores, high drug loading capacity, customizable shape and pore size, surface modifiability, and biocompatibility.^{[16][16]} Inulin, a fructose polymer composed of D-fructose units and widely occurring in nature^{[17][17]}, has achieved generally recognized as safe (GRAS) status and is recognized as a green prebiotic food.^{[18][18]} Moreover, inulin has great potential to be developed as an innovative colon-targeting carrier due to its unique metabolic process in humans: it resists absorption in the upper GIT and exclusively undergoes fermentation through the action of inulinase, which is presented solely in the colon.^{[19][19]} In addition, inulin can be used as a prebiotic to promote the proliferation of beneficial bacteria (i.e. Bifidobacterium and Lactobacillus) and maintain intestinal micro-ecological balance and host health, and has been shown to have favorable benefits in the management of IBD.^{[20][20]} Inulin acted as a “gate-keeper” when coated onto the surface of the mesoporous silica core to control the release of drugs using a “switch on-switch off” procedure as exemplified herein (FIG. 1A).

In this study, use of the inulin layer as a “gate-keeper” played a dual role, not only as a trigger for drug release but also in supporting the gut microbiota homeostasis, making it a key and innovative component of the nanoparticles. As shown in FIG. 1B, after oral administration, the nanoparticles remained stable when passing through the upper GIT. Upon encountering inulinase produced by the colon microflora, the inulin within the prepared prebiotic nanoparticles disassembled, triggering the selective release of Bud in the colon, thereby maximizing its anti-inflammatory function. These findings indicate that inulin improved the efficiency of Bud delivery owing to its property of degrading in the colon. Meanwhile, serving as a prebiotic, inulin can regulate the gut microbiota, fostering the elimination of inflammation. In summary, this study offered a precise oral strategy for colon-targeting therapy to attenuate IBD based on a natural prebiotic-mediated nanoparticles. These nanoparticles exhibited outstanding colon triggered “switch on” drug release, effective anti-inflammatory effect, and regulation of intestinal flora, revealing potential perspectives for inulin-based colon targeting therapy in IBD and other colon diseases.

### Results

### Design, Preparation and Characterization of the Nanoparticles

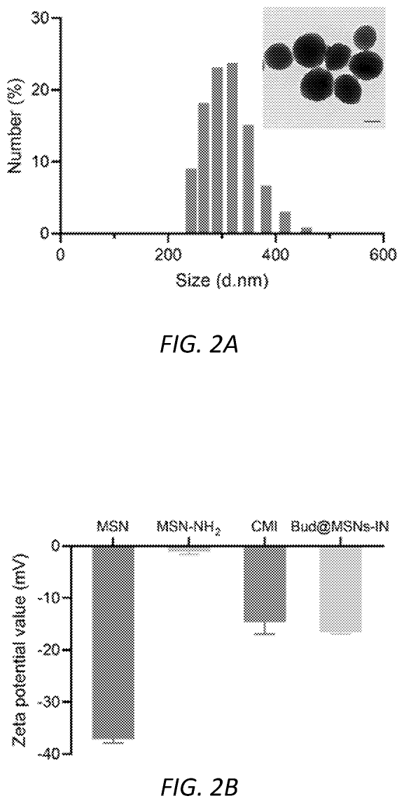

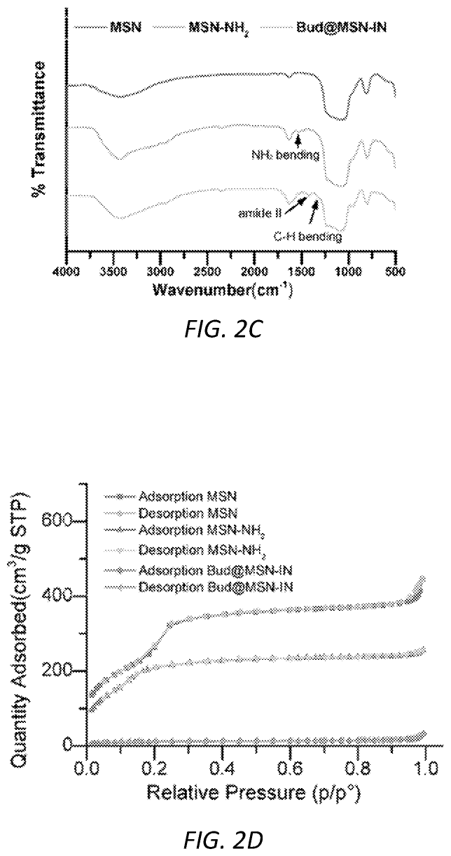

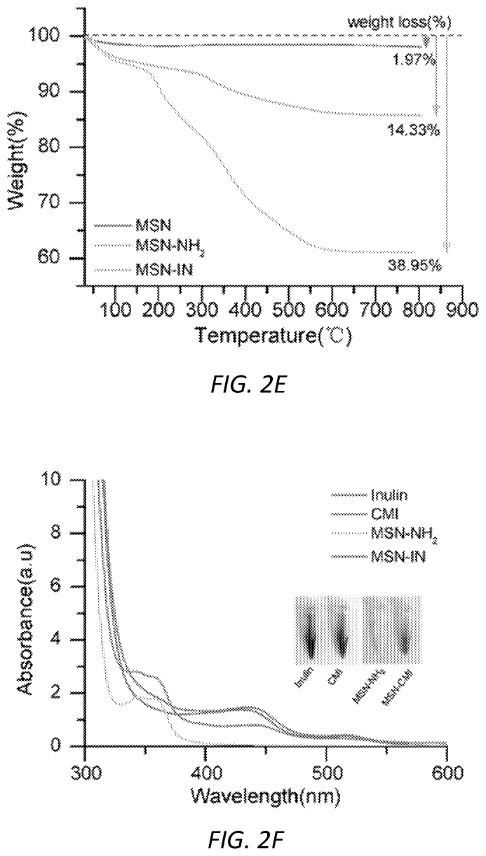

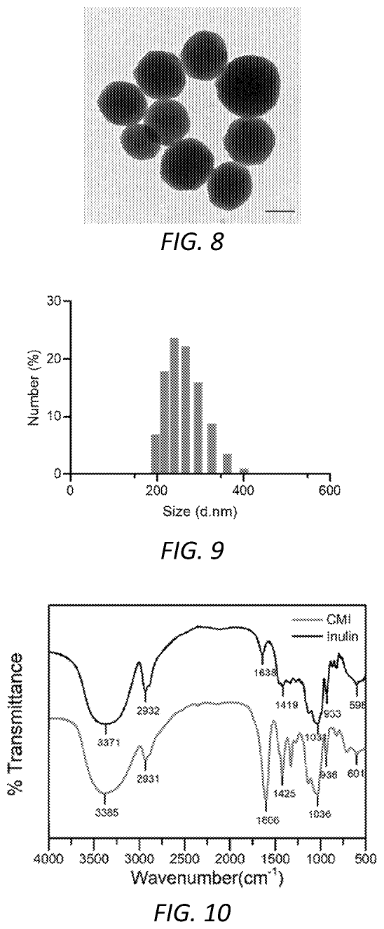

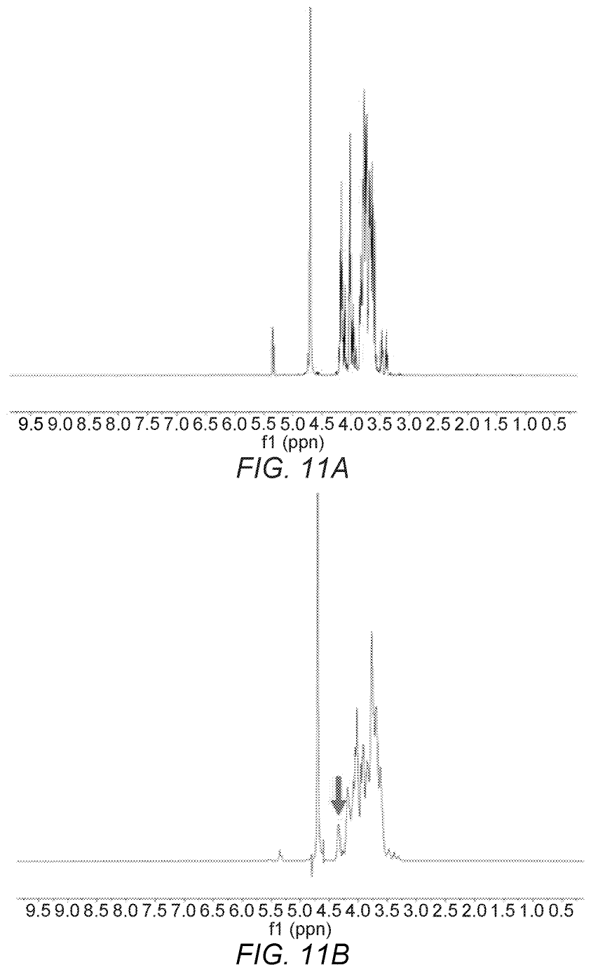

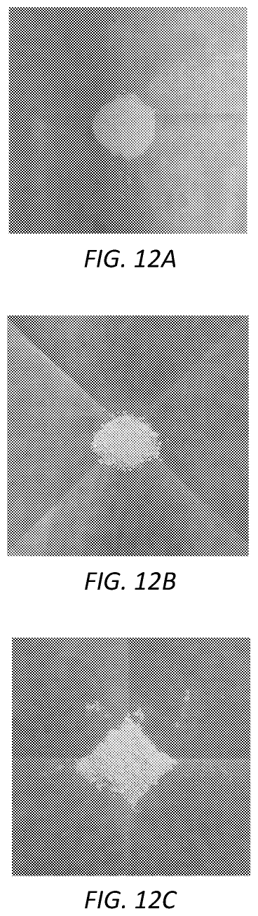

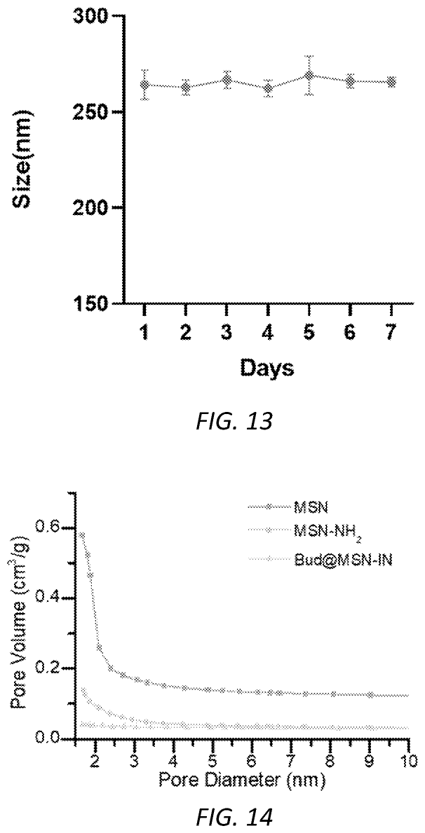

Nanoparticles were designed and prepared to release cargo drug molecules in response to inulinase using three key components: (1) an MSN core as the cargo carrier; (2) the budesonide payload was encapsulated in the pores of MSNs by physisorption; and (3) inulinase-reactive inulin was coated on the surface of MSNs through an amide reaction to protect the drug from being released in the upper GIT, thereby allowing its release in the colon (FIG. 1A). The prepared MSNs had a uniform particle size of 244.3±7.2 nm and zeta potential of −37.2±0.70 mV (FIGS. 2B, 8, and 9). Carboxymethyl inulin (abbreviated as CMI) was synthesized by carboxymethylation of the carboxyl side chains on inulin. The attachment of carboxymethyl groups to inulin was confirmed by Fourier transform-infrared spectroscopy (FT-IR) and nuclear magnetic resonance (NMR) spectroscopy. In the FT-IR spectrum (FIG. 10), the peaks for the inulin at 3300-3500 cm⁻¹, 1638 cm⁻¹ and 1031 cm⁻¹ respectively indicated O—H stretching, O—H bending and C—O—C bending. After the coupling reaction, it was clear that the O—H stretch band of inulin diminished when the —OH groups were substituted by the carboxymethyl groups, while two strong peaks emerged at 1606 cm⁻¹ and 1425 cm⁻¹ (in the CMI spectrum). These peaks can be ascribed to asymmetrical and symmetrical stretching of the COO— group^{.[21][21]} In ¹H NMR, the chemical shifts of inulin were a narrow region ranging from 3.20-5.50 ppm, appearing a typical characteristic of polysaccharide (FIG. 11A).^{[22][22]} It is noted from FIG. 11B, a new peak at 4.3 ppm was observed in modified inulin, indicating the introduction of carboxymethyl groups at O-6 position.^{[23][23]} To conjugate CMI to the surface of MSNs, the inner and outer surfaces of MSNs were firstly functionalized with amine groups by post-grafting the silica surface with (3-aminopropyl)triethoxysilane (APTES) (designated as MSN-NH₂). Then, after loading budesonide into the pores of the MSNs, the prepared nanoparticles (designated as Bud@MSN-IN) were synthesized via the amide reaction between the —COOH group of CMI and the —NH₂ group of MSN-NH₂ in the presence of EDC/NHS (FIG. 2C). The resultant Bud@MSN-IN NPs were observed to be light yellow in appearance and spherical in shape, with a hydrodynamic particle size of 276.7±14.4 nm and zeta potential of −16.6±0.4 mV (FIGS. 2A, 2B, and 12A-12C). There was no significant change in particle size of Bud@MSN-IN NPs stored in water for 7 days, suggesting its good stability (FIG. 13).

Amine functionalization and CMI coupling to MSNs were confirmed by zeta potential, FT-IR, N₂ adsorption/desorption isotherms and the use of thermogravimetric analysis (TGA). Compared to as-synthesized MSNs (930.65 m²/g in Brunauer-Emmett-Teller (BET) surface area, 0.58 cm³/g in pore volume), both MSN-NH₂ and Bud@MSN-IN showed a decrease in BET surface areas (796.76 and 38.08 m²/g, respectively) under total pore volumes (0.14 and 0.04 cm³/g, respectively) (FIGS. 2D and 14, and Table 1).

TABLE 1
Brunauer-Emmett-Teller (BET) surface area and total
pore volume of MSN, MSN-NH₂, Bud@MSN-IN NPs analyzed
from the N₂ adsorption/desorption isotherms.

| | Surface area | Pore volume |
|---|---|---|
| Sample | S_{BET} (m²/g) | Vp (cm³/g) |
| MSN | 930.65 | 0.58 |
| MSN-NH₂ | 796.76 | 0.14 |
| Bud@MSNs-IN | 38.08 | 0.04 |

These results further confirmed the sequential grafting of APTES and the CMI. The organic content of nanoparticles was estimated as a weight percentage via TGA. As shown in FIG. 2E, weight loss in the TGA trace was attributed to the pyrolysis of organic contents, and the residue was assumed to be noncombustible silica. A weight loss of 12.36% and 24.62% was observed between the amino-functionalized and bare silica nanoparticles, CMI coupling-functionalized and amino-functionalized nanoparticles, respectively. The loading rates of inulin and budesonide in Bud@MSN-IN NPs were 23.54±1.37(%) and 14.47±0.60(%), respectively (Table 2).

TABLE 2
The drug loading rate of Bud@MSN NPs and Bud@MSN-IN
NPs (mean ± SD, n = 3).

| | Bud@MSN NPs | Bud@MSN-IN NPs |
|---|---|---|
| Bud (%) | 23.34 ± 0.91 | 14.47 ± 0.60 |
| Inulin (%) | — | 23.54 ± 1.37 |

Furthermore, MSN-IN appeared to be brick-red color under the thiobarbituric acid reaction, which was similar to inulin and CMI. The UV-vis absorption spectra of MSN-IN had two characteristic inulin maximum absorption peaks at 450 nm and 530 nm (FIG. 2F). These above results indicated that these functional groups were successfully coupled to the MSNs.

### In Vitro Drug Release Studies

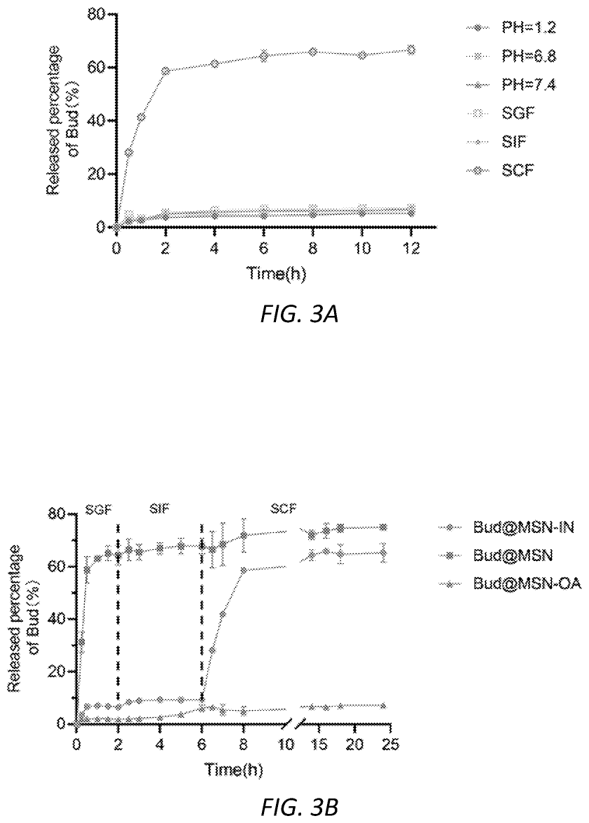

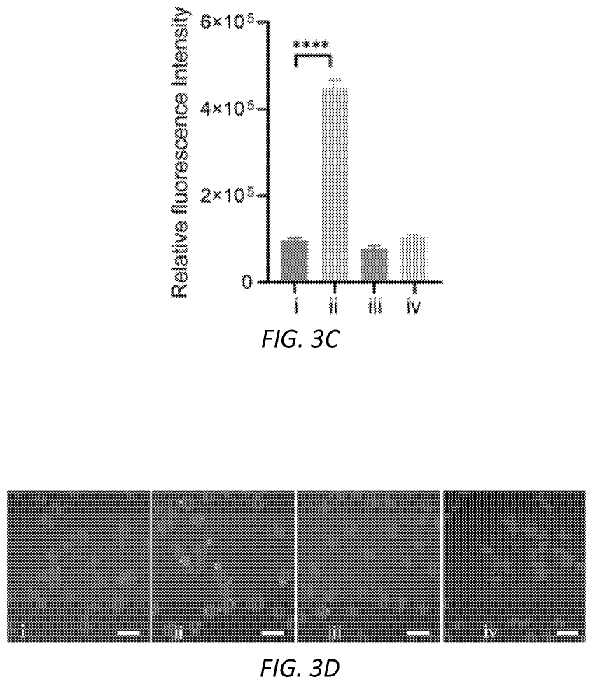

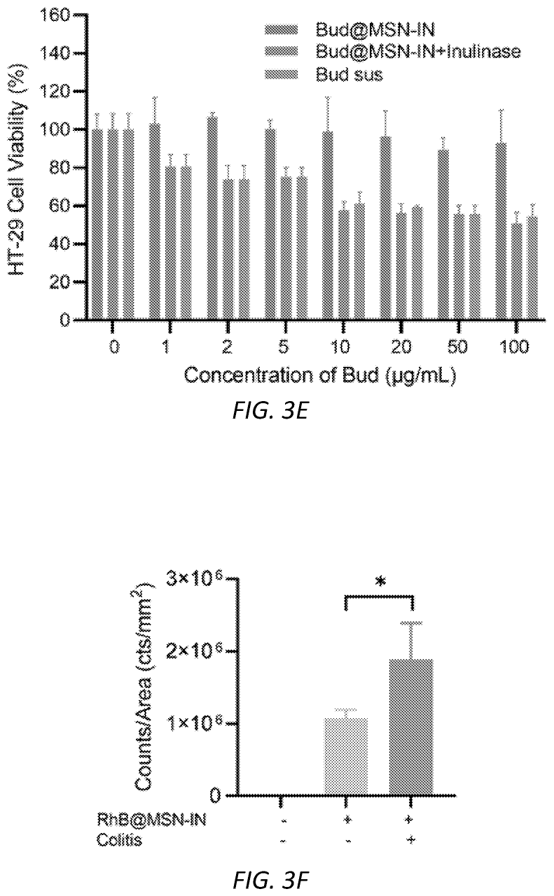

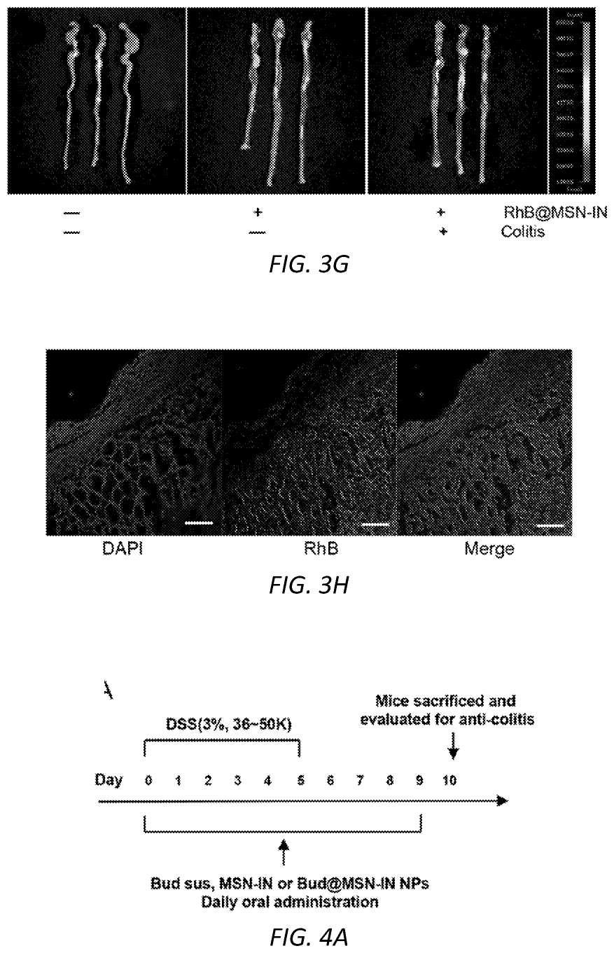

To verify the colonic enzyme-triggered “switch on” drug release property of Bud@MSN-IN NPs, the in vitro drug release curves of Bud@MSN-IN NPs were investigated under different pH levels and enzymatic environments. As shown in FIG. 3A, the results showed that the release of Bud from the formulation was less than 10% in different pH media as well as in the enzymatic environment mimicking stomach and small intestine, whereas at simulated colon enzymatic media, more than 60% of Bud could be released within 2 hours (h). Thereafter, the in vitro drug release profiles were further compared for Bud@MSN without inulin coating, Bud@MSN-IN, and Bud@MSN-OA with non-inulinase responsive oleic acid coating by exposing these nanoparticles to simulated intestinal fluids in different parts of the GIT system. Considering both mean residence times and physiological conditions, these nanoparticles were incubated successively with (1) simulated gastric fluid (SGF, pepsin enzyme, pH 1.2) for 2 h, (2) simulated intestinal fluid (SIF, pancreatin enzyme, pH 6.8) for 4 h, and (3) simulated colonic fluid (SCF, inulinase enzyme, pH 7.4) for more than 18 h.^{[24][24]} The results showed that the Bud@MSN NPs without inulin coating released a large amount of Bud in SGF, with 60% of Bud being released within 30 minutes (min). In release curve of Bud@MSN-IN NPs, there was only a small amount of budesonide (less than 7%) released during the first 2 h of incubation in SGF. Then, after the buffer was changed to SIF, minimal Bud was released, not exceeding 10%. When the medium was changed to SCF, a burst release was observed, with an additional 20% of the entrapped drug liberated after 30 min of incubation in the presence of inulinase. After 24 hours of incubation, approximately 65% of the drug was released in the simulated gastrointestinal environment (FIG. 3B). In contrast, the Bud@MSN-OA nanoparticles did not exhibit inulinase-responsiveness and were unable to release cargo drugs, suggesting the key and crucial role of inulin in the colon-targeting drug delivery of the nanoparticles. These results indicated that inulin could prevent the premature release of the loaded drug as the nanoparticles passed through the upper GIT. Upon encountering inulinase, which is presented solely in the colon, the inulin disassembled through the hydrolysis of linear β-2,1-linked fructose chain. As a result, the gates of the mesopores were detached, allowing the entrapped cargo to be released from the MSNs. Taken together, these data suggest that the inulin-mediated nanoparticles can be used as colon targeted “switch on” drug delivery system.

### Colon-Targeting Performance of the Nanoparticles

Cytocompatability is a primary concern in the efforts to develop new oral drug delivery systems. To evaluate the cytotoxicity of these nanoparticles, HT-29 cells were treated with various concentrations of these nanoparticles. The results in FIG. 16 revealed that both MSN-IN NPs and Bud@MSN-IN NPs showed good cytocompatibility. The cytotoxicity effect of Bud@MSN-IN NPs was then compared at different concentrations of contained Bud with and without inulinase (5 mg/mL). The free drug of Bud served as a positive control to demonstrate the safety and inulinase responsiveness of Bud@MSN-IN NPs. After co-incubation with inulinase for 24 h, Bud was released from Bud@MSN-IN NPs, thus producing a similar cytotoxicity effect to free Bud on HT-29 cells (FIG. 3E). Moreover, in order to explore the endocytosis of nanoparticles by Caco-2 cells, confocal laser scanning microscopy (CLSM) images were taken after the prepared MSN-IN NPs were labelled with Rhodamine B (RhB@MSN-IN) NPs. As shown in FIGS. 3C and 3D, strong fluorescence signal was observed in the group of RhB@MSN-IN NPs+inulinase after 18 h incubation, whereas negligible fluorescence was observed for the groups of RhB@MSN-IN NPs in the absence of inulinase. Furthermore, negligible fluorescence was observed in the RhB@MSN-OA NPs groups with or without inulinase. These results confirmed that the nanoparticles can act as biocompatible nanocarriers that are responsive to inulinase and are thus capable of selectively delivering drug payloads to the colon cells.

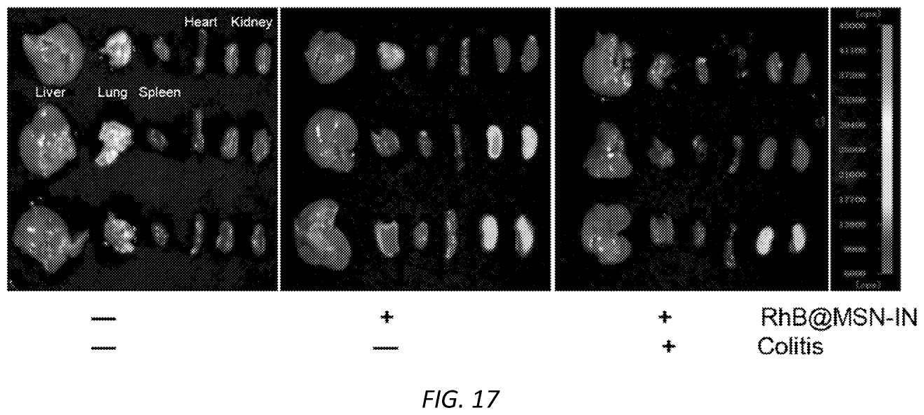

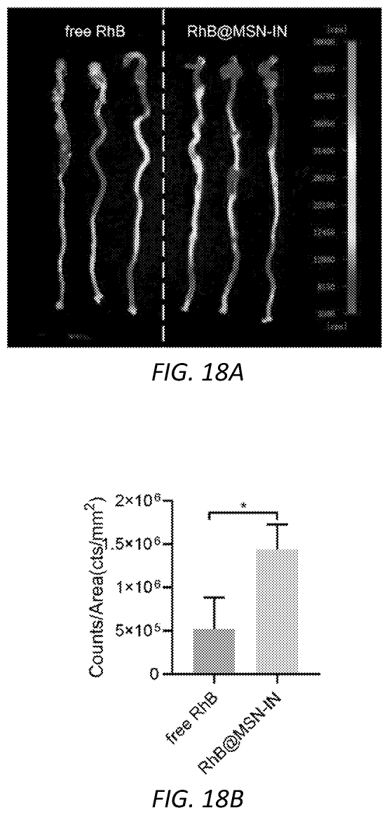

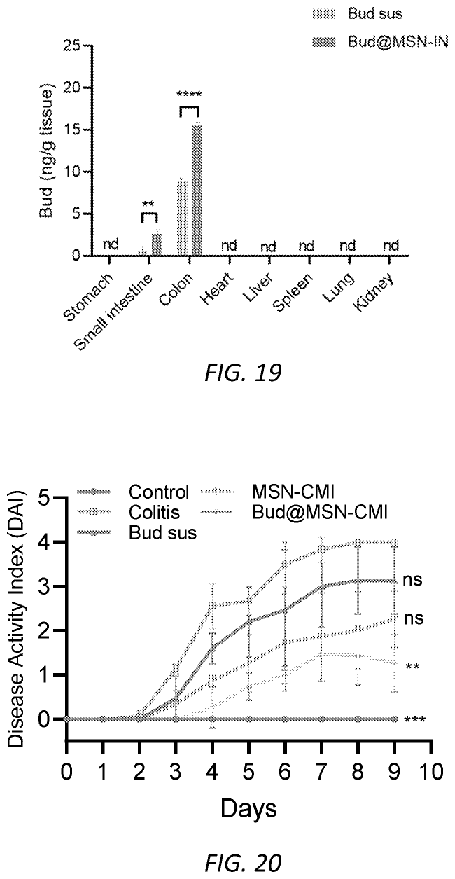

The selective accumulation of nanoparticles in colon is one of the most important issues in the effective therapy of IBD. To investigate whether the prepared nanoparticles preferentially adhered to the inflamed mucosa in vivo, the biodistribution of RhB@MSN-IN NPs in both healthy mice and DSS-induced acute colitis model mice was studied. After the oral administration of RhB@MSN-IN NPs for 6 h, only slight fluorescence was detected in the colon of healthy mice, indicating a small amount of accumulation of RhB@MSN-IN NPs in the colon tissue. In contrast, a considerable accumulation of the RhB@MSN-IN NPs was observed in the colonic tissue of colitis mice (FIGS. 3F and 3G). At the same time, only slight fluorescence emission was found in the major organs (heart, kidneys, liver, lung, and spleen) of colitis mice, indicating that the RhB@MSN-IN NPs were largely localized to the inflamed colon (FIG. 17). CLSM was used to observe the cellular localization and distribution of RhB@MSN-CMI NPs in colon tissue. As shown in FIG. 3H, clear red fluorescence was observed in the epithelial layer as well as in the lamina propria under the epithelium, indicating that these nanoparticles were distributed in the colonic epithelial layer and the underlying lamina propria. In addition, in the healthy mice, a higher fluorescence signal of RhB@MSN-IN NPs accumulated in the colon than that in the free RhB group (FIGS. 18A and 18B). This may be associated with the nanometer size of the nanoparticles and the strong adhesion of the intestinal epithelium that increased their residence time in the colon. Collectively, these results demonstrated that RhB@MSN-IN NPs can selectively accumulate in the inflamed colon, with a targeting efficiency that was significantly higher than that of the control-free RhB molecule. Furthermore, the biodistribution of budesonide was determined in colitis mice 24 hours after oral administration of Bud@MSN-IN and free Bud sus using HPLC. As shown in FIG. 19, Bud was not detected in any of the investigated organs except for the small intestine and colon. Compared to colon tissue, only a very small amount of Bud was found in the small intestine tissue. In addition, the Bud levels in the colon of Bud@MSN-IN NPs treated group were significantly higher than that of the Bud sus group. The above results indicated that, by packaging the payload molecules into MSN-IN NPs, they can be preferentially localized into the colitis tissue, thereby decreasing their indiscriminate accumulation in healthy organs and maximizing bioavailability at disease sites.

### Therapeutic Efficacy of the Nanoparticles in a DSS-Induced Acute Colitis Model

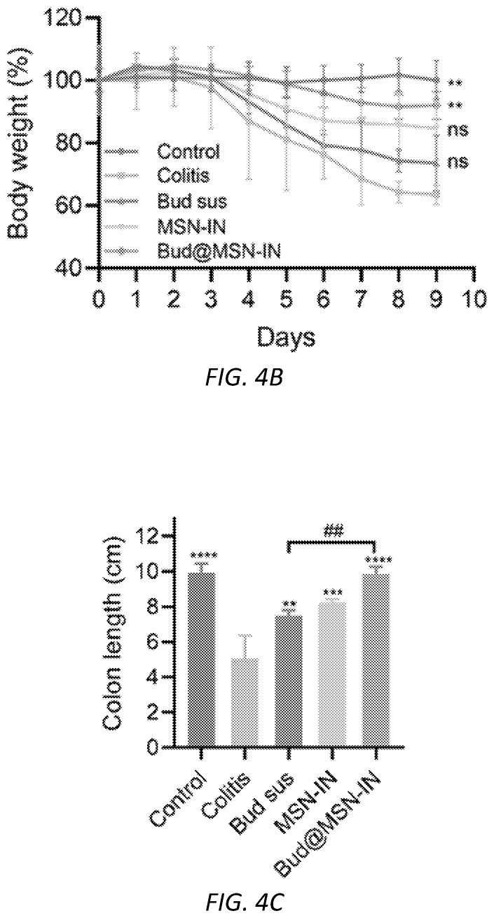

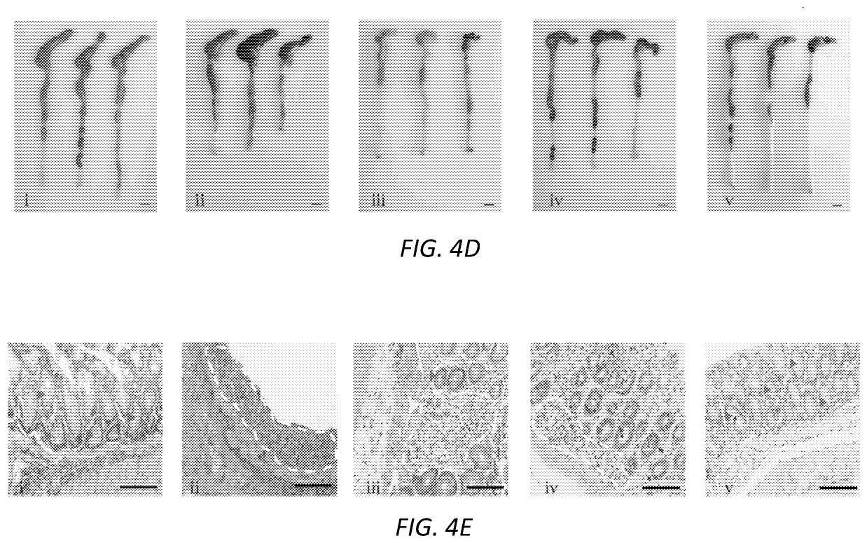

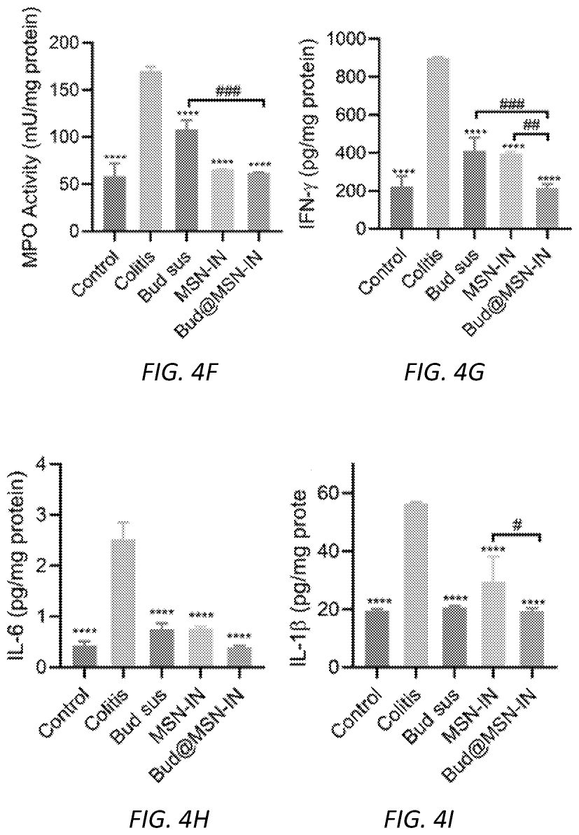

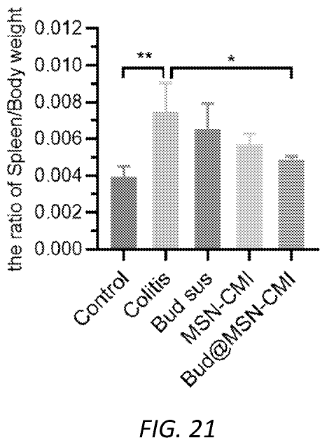

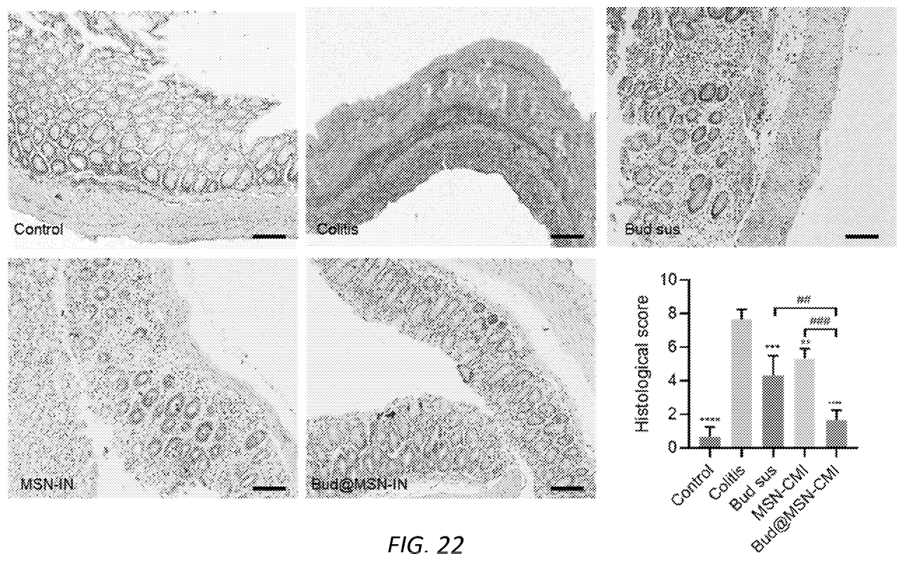

The efficacy of Bud@MSN-IN NPs was evaluated in treating DSS-induced colitis mice. In this experiment, the mice were randomly divided into five groups and subjected to different treatment modalities for 10 days (FIG. 4A). The body weight of all DSS-treated mice gradually decreased during the experiment, while therapy with Bud sus, MSN-IN NPs, and Bud@MSN-IN NPs was able to mitigate weight loss. The Bud@MSN-IN NPs-treated group showed minimal weight loss (FIG. 4B). After treatment, the severity of colitis was further assessed. The results showed that both decreased colon length and increased disease activity index (DAI) in the colitis mice were modulated by both Bud sus and Bud@MSN-IN NPs. The latter treatment reversed the values to a greater extent compared to the free drug, resulting in a colon morphology comparable to that of the healthy control (FIGS. 4C, 4D, and 20). Meanwhile, Bud@MSN-IN NPs had the greatest effect with respect to reducing the increase in spleen-to-body weight ratio caused by inflammation (FIG. 21). Further examination of histological sections revealed that the inflamed colon of DSS-treated mice showed serious necrocytosis, significant loss of goblet cells (red arrow), and destruction of crypt structures (green arrow). The mucosa and submucosa of mice with DSS-induced colitis were notably infiltrated in large areas by inflammatory cells (yellow circles). After treatment with Bud@MSN-IN NPs, significant recovery from this pathological damage was observed, and the tissue is more closely resembled that of normal mice. The goblet cell levels returned to normal, and the edema disappeared, with no inflammatory cell infiltration observed. In contrast, moderate recovery from pathological damage was found in the colon in the Bud sus and MSN-IN NPs groups (FIGS. 4E and 22).

To further investigate the mechanism by which Bud@MSN-IN NPs alleviates colitis in mice, the expression levels of oxidative stress-related and pro-inflammatory mediators in colonic tissues were examined. In experimental colitis, disease severity was always found to be correlated with myeloperoxidase (MPO) activity, a glycosylated enzyme found in the granules of neutrophils and monocytes. Thus, this enzyme serves as a biomarker for assessing disease status in IBD patients.^{[25][25]} As shown in FIG. 4F, MPO activity was substantially increased in the colitis group, while this increase was significantly attenuated by the treatment with Bud sus, MSN-IN NPs and Bud@MSN-IN NPs, in which the Bud@MSN-IN NPs group showed the most reduction. This indicated that treatment with Bud@MSN-IN NPs may substantially inhibit neutrophil infiltration into the inflamed colon. DSS treatment led to increased levels of several proinflammatory cytokines, including interferon gamma (IFN-γ, FIG. 4G), interleukin-6 (IL-6, FIG. 4H), and IL-1β (FIG. 4I). Such upregulation was consistent with the results seen in previous studies.^{[26][26]} Reduction in inflammatory cytokine levels in serum is a logical target for IBD therapy. Both Bud sus and MSN-IN NPs significantly contributed to inhibition of DSS-induced elevation in the proinflammatory cytokine contents in the colon tissues. Treatment with Bud@MSN-IN NPs significantly decreased the levels of proinflammatory cytokines such that they were comparable to mice not treated with DSS. Thus, the above results indicated that Bud@MSN-IN NPs could serve as a precise strategy for the colon-targeted treatment of IBD.

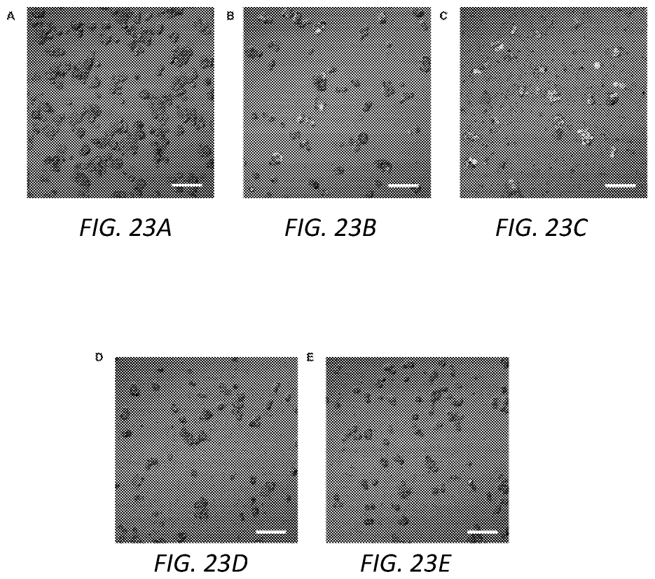

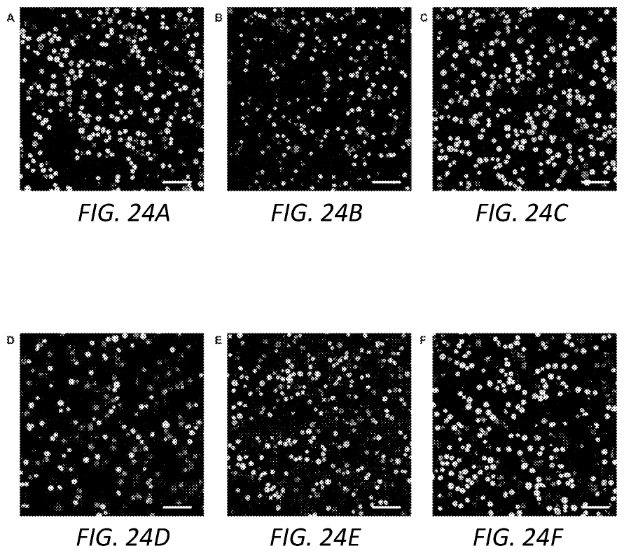

MSN-IN also has a certain anti-inflammatory effect in DSS-induced colitis mice. This may be due to the fact that inulin is known to have antioxidant properties.^{[27][27]} As shown in FIGS. 23A-23E, under the equivalent Bud+MSN treatment conditions, treatment with inulin-coated NPs (MSN-IN, Bud@MSN-IN) significantly reduced the levels of intracellular ROS in HT-29 cells, indicating that the inulin-coated nanoparticles have strong antioxidant effects. The protective effect of these nanoparticles for HT-29 cells was explored using H₂O₂ as an oxidative toxic chemical control. AM/PI assays showed that cell proliferation was substantially reduced in H₂O₂-treated cells but was able to be significantly rescued in the presence of inulin-coated nanoparticles or free inulin (FIGS. 24A-24F). These findings clearly indicate that, by virtue of their intrinsic high antioxidant power, MSN-IN and Bud@MSN-IN NPs can protect cells from oxidative damage, something that is consistent with the reported results.^{[28][28]}

### Restoration of Colonic Epithelium

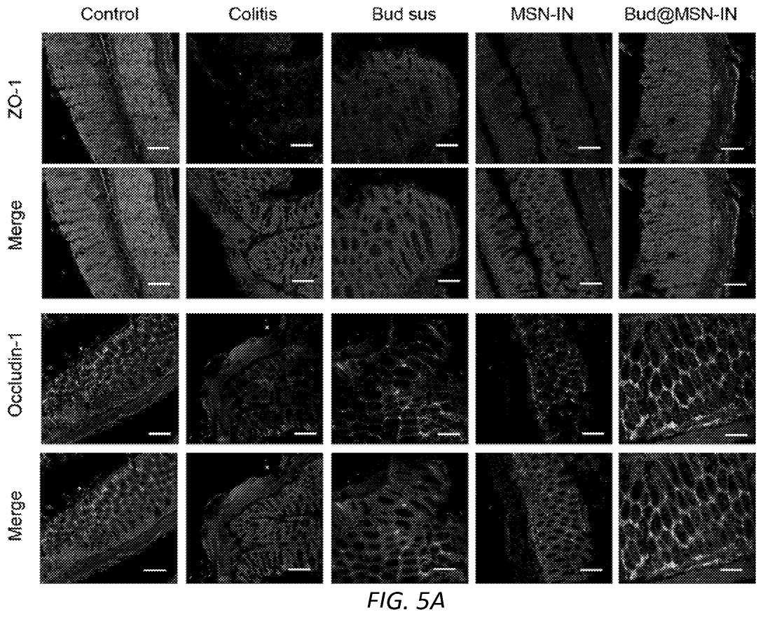

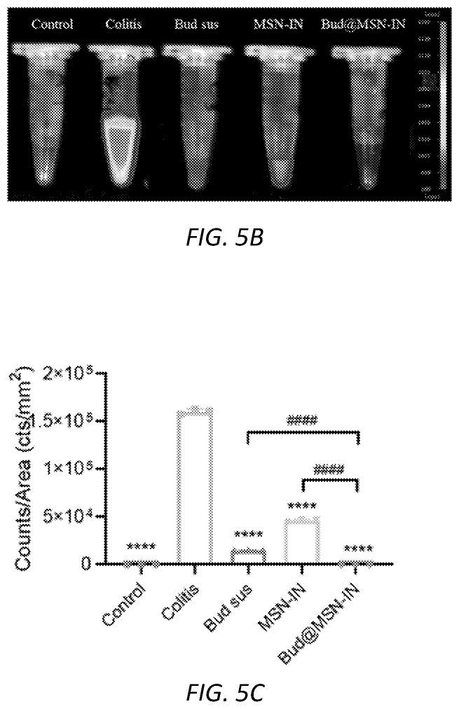

Colitis also involves disruption of the intestinal epithelial barrier. This damage may allow bacterial translocation and the entry of other antigens, eventually resulting in recurrent inflammation.^{[29]} The mice treated with DSS also showed considerably decreased levels of tight junction-associated proteins zonula occludens-1 (ZO-1) and Occludin-1 (FIG. 5A). These factors play a pivotal role in gut homeostasis.^{[30]} In contrast, the colitis mice treated with Bud@MSN-IN NPs showed a significant increase in ZO-1 and Occludin-1 levels relative to those in the DSS group; however, other groups, including Bud sus and MSN-IN treatment, also had a minimal impact on ZO-1 and Occludin-1 levels. Intestinal barrier function was evaluated using in vivo FITC-dextran penetration tests. Compared to other treatments, orally administrated Bud@MSN-IN NPs was also able to prevent systemic exposure of FITC-dextran (4 MW) into DSS-colitis mice (FIGS. 5B and 5C). This result demonstrated that Bud@MSN-IN NPs were able to help recover epithelial cell injury and restore barrier integrity.

### Modulation of Gut Microbiome Composition

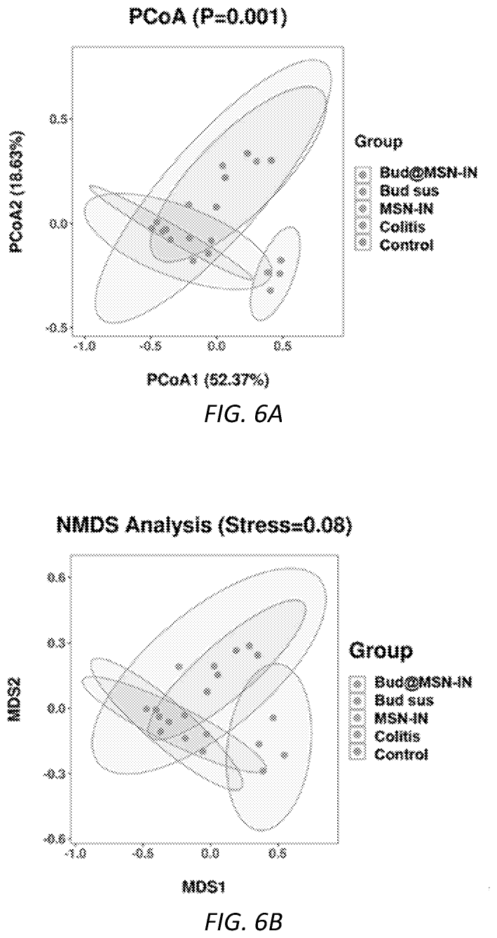

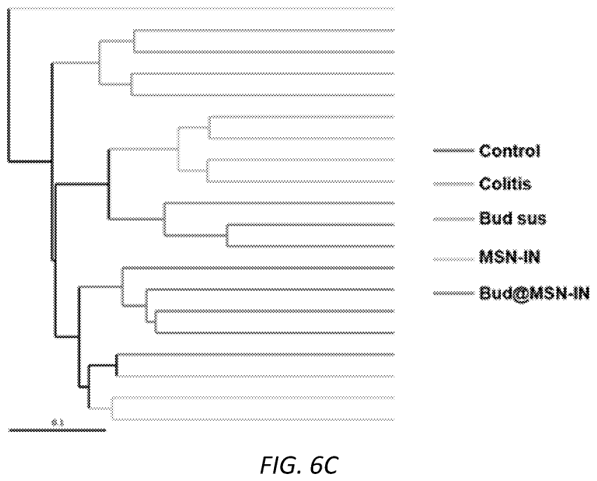

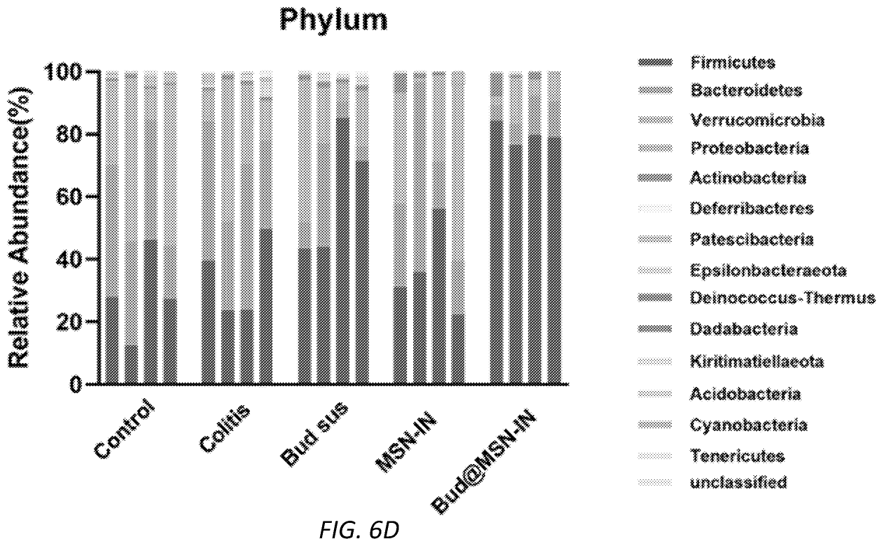

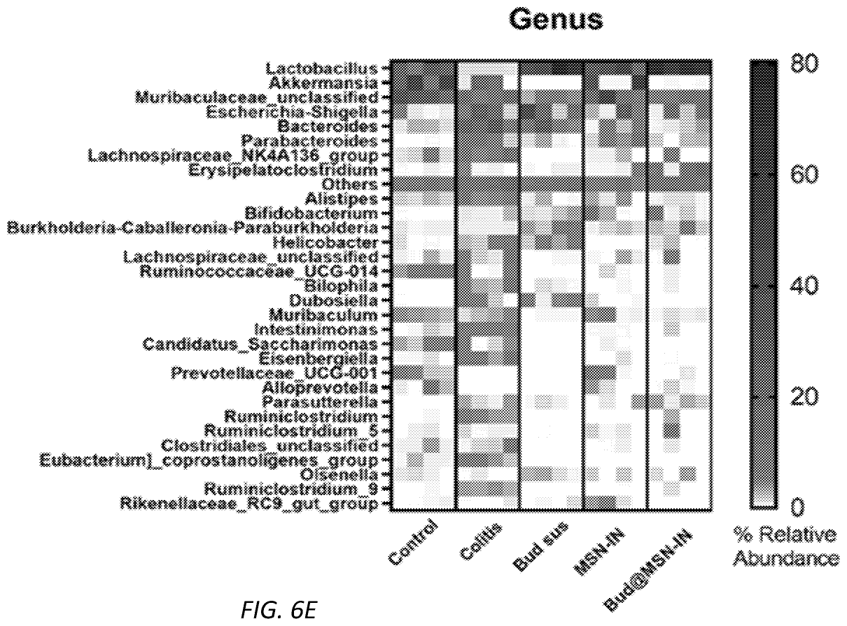

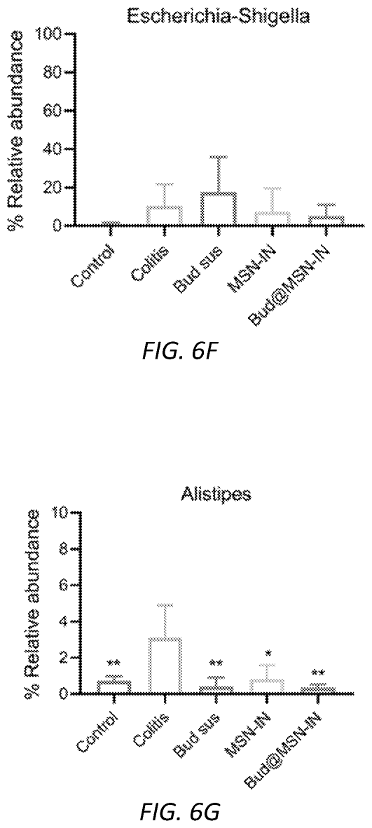

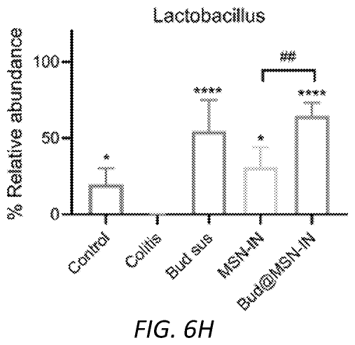

Multiple studies have shown that inflammatory diseases of the gastrointestinal tract are frequently associated with changes in gut microbial communities.[31][31] Moreover, as a recognized prebiotic, inulin is specifically fermented by colon bacteria, stimulating the proliferation of beneficial bacteria (i.e., Bifidobacterium and Lactobacillus), controlling pathogens (E. coli and clostridia) at low levels and maintaining gut microbial balance.[32][32] In order to further explore the potential anti-IBD mechanisms, it was investigated whether the oral administration of Bud@MSN-IN NPs could also modulate the composition of the gut microbiota in colitis mice. In this regard, analysis of beta diversity distance matrix using principal coordinate (PCoA) and nonmetric multidimensional scaling (NMDS) showed that there were significant changes in the overall structure of the gut bacterial community after DSS treatment (FIGS. 6A and 6B), and this result was consistent with a previous finding on the DSS-induced mice model.[33][33] Interestingly, treatment with Bud sus and Bud@MSN-IN NPs drove the gut microbiota away from that of the DSS group, bringing it close to the level of the normal group. An evident pattern shift along with both PCoA1 and PCoA2 was observed in the MSN-IN NPs treatment group. This pattern overlapped with that of the control group. The system clustering tree also revealed a drastic difference in fecal microbiota between the five treatment groups, where Bud@MSN-IN NPs treated samples were clustered separately from the colitis group (FIG. 6C).

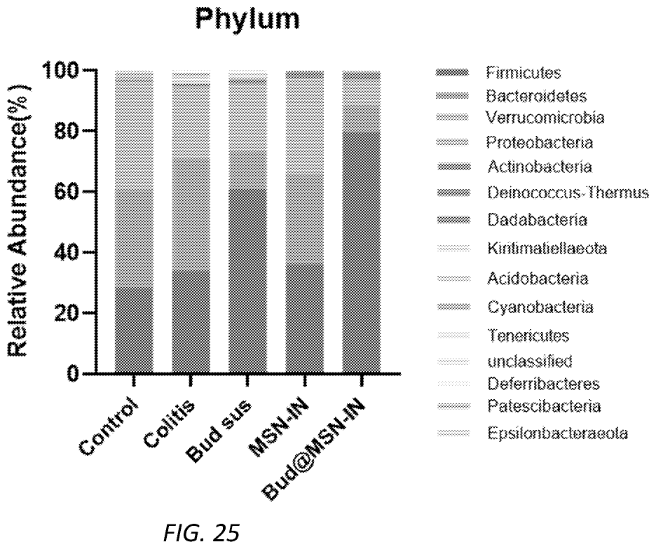

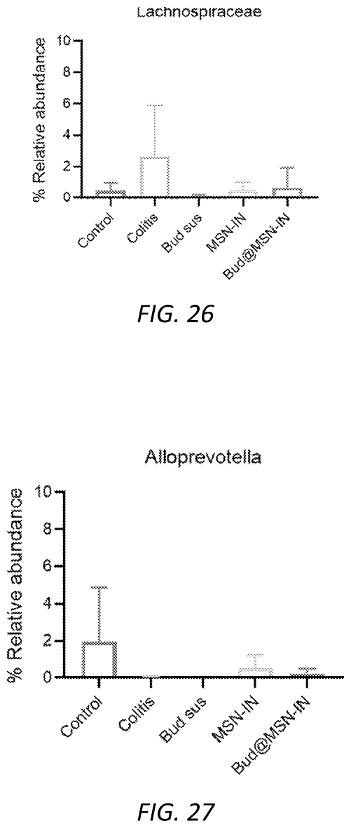

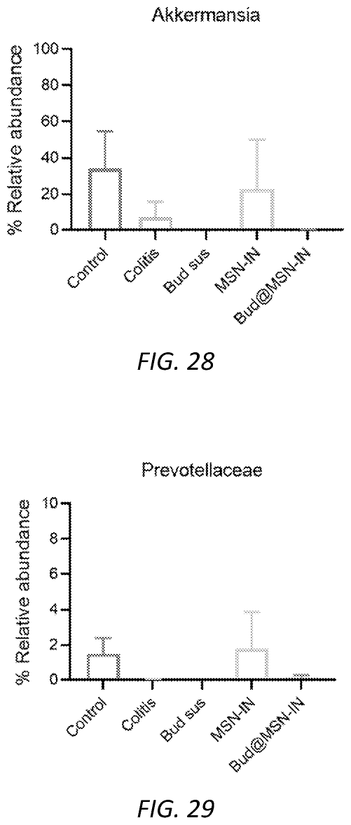

Further analysis at the phylum level showed that the fecal microbiota of the healthy mice was dominated by Firmicutes, Bacteroidetes, Verrucomicrobia and Proteobacteria phyla (FIGS. 6D and 25). The fecal bacterial of IBD patients has been reported to be different from that of healthy individuals, with a higher ratio of pathogenic bacteria to commensal flora, (Escherichia coli, Campylobacter spp., Mycobacterium avium) (Bacteroides and Firmicutes phyla) and a decreased bacterial load in areas of active inflammation.[34][34] Here, both of the Bacteroidetes and Proteobacteria dramatically increased when the mice were fed with DSS (FIGS. 6D and 25). Compared to healthy control mice, the colitis mice showed a markedly increased proportion of Escherichia-Shigella (FIGS. 6E and 6F), well-known infective pathogens that contribute to the development of colitis.[35][35] Moreover, the growth of conditional pathogenic bacteria Alistipes (FIG. 6G) and Lachnospiraceae (FIG. 26) at a genus level increased as the microbial drivers of colitis when the mice were fed with DSS. In contrast, Bud@MSN-IN NPs treatment significantly reduced the relative abundance of pathogenic bacteria and increased sequence types representative of beneficial Alloprevotella (FIG. 27) which can produce short-chain fatty acids for the protection of the gut barrier and Lactobacillus (known for beneficial roles in both IBD animal models[36][36] and IBD patients[37][37]). Treating the mice with colitis with MSN-IN NPs significantly increased the relative abundance of Akkermansia (known to be associated with protective intestinal barrier functions[38][38]) and Prevotellaceae (known to exert anti-inflammatory effects by producing SCFAs) (FIGS. 28 and 29). The results suggested that the Bud@MSN-IN NPs were able to reverse DSS-induced changes in the gut microbiota of colitis mice, likely due to the prebiotic function of inulin.

### Safety Evaluations of the Nanoparticles for Oral Administration

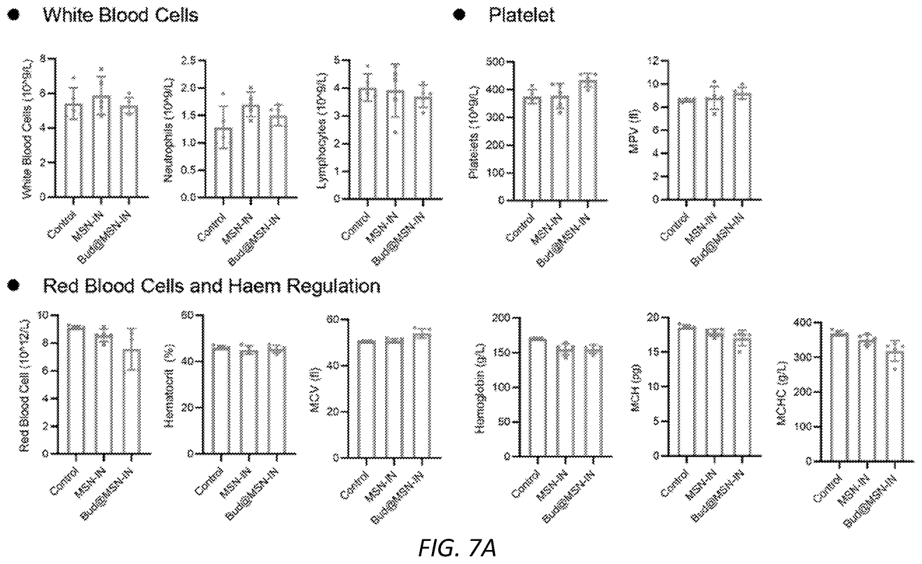

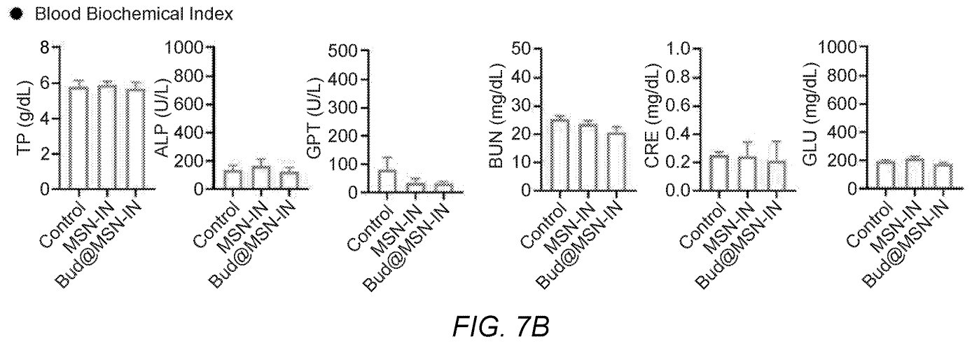

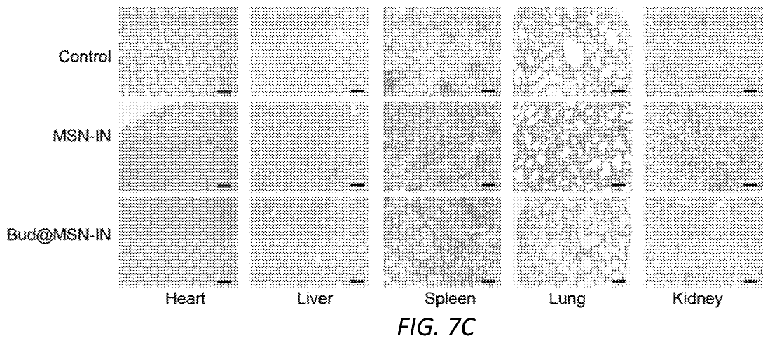

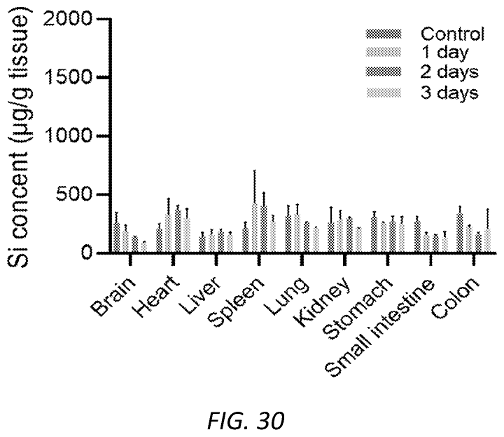

Since the above findings demonstrated that prepared nanoparticles show promise for the targeted therapy of colitis, the safety profile for oral delivery was evaluated. MSN-IN and Bud@MSN-IN NPs were respectively orally administered in mice for 10 days. After such administration, no animal deaths occurred due to toxicosis at all of the dose levels examined. At day 10, the mice were euthanized. Representative hematological parameters were found in the normal ranges for mice treated with MSN-IN or Bud@MSN-IN NPs (FIG. 7A). Quantification of biomarkers indicative of hepatic and kidney functions (including TP, ALP, GPT, GLU, BUN, and CRE) revealed no abnormal variations in the MSN-IN and Bud@MSN-IN NPs treated group compared to the control group (FIG. 7B). Consistent with these results, further examination of H&E sections of major organs showed that repeated administration of MSN-IN or Bud@MSN-IN NPs for 10 days caused no discernable injuries compared to healthy control mice (FIG. 7C). All of these results suggested that both MSN-IN and Bud@MSN-IN NPs exhibited excellent safety profiles via oral administration. Furthermore, the excretion kinetics of silica nanoparticles was evaluated by measuring silicon levels in the gastrointestinal tissues and major organs. The ICP results showed that after 1, 2 or 3 days of administrated Bud@MSN-IN NPs, there was no statistically significant difference in the silicon content of different organs compared to the control group (FIG. 30), indicating that these nanoparticles have good biosafety and can be completely cleared from the body in less than 1 day.

### Methods and Materials

The following materials and methods were used in the Examples herein.

### Materials

All chemicals were at least of analytical grade and used without further purification. N-Cetyltrimethylammonium bromide (CTAB, 98%), tetraethyl orthosilicate (TEOS, 98%), (3-Aminopropyl)triethoxysilane (APTES, 99%), N-(3-Dimethylaminopropyl)-N′-ethylcarbodiimide hydrochloride (EDC, 98%), N-Hydroxysuccinimide (NHS, 98%) Budesonide, Inulin, monochloroacetic acid, anhydrous methanol, acetonitrile, hydrochloric acid, Sodium hydroxide (AR, 96%) were bought from Aladdin Reagent Co. Ltd. (Shanghai, China) and used as received. Ultrapure water (Hangzhou Wahaha Group Company Ltd, Hangzhou, China) was used throughout. All other reagents were obtained by Tianjin Institute of Urology (China) and used without further purification.

### Synthesis of the Bud@MSN-IN NPs

Synthesis and Amination of mesoporous silica nanoparticles (MSNs): Mesoporous silica nanoparticles (MSNs) were synthesized using a sol-gel reaction in the presence of cationic surfactant templates.[42][42] Briefly, CTAB (1.00 g, 2.74×10⁻³ mol) was dissolved in a mixture of deionized water (500 mL) and NaOH aqueous solution (2.00 M, 4 mL). The solution was then heated to 80° C. and allowed to stir for 30 min. TEOS (5.00 mL) was added dropwise into the solution, and the mixture was stirred for another 2 h at 80° C. to form a white suspension. The white precipitate was filtered off, washed with methanol, and dried in a vacuum oven at 45° C. Finally, MSNs were obtained upon removal of CTAB template by calcination at 550° C. for 6 h.

To functionalize amine groups on the surface of MSNs, MSNs (500 mg) were dispersed in 50 mL of anhydrous methanol then APTES (1.50 mL) was added to the above solution. The reaction was allowed to proceed at room temperature for 12 h. The prepared amino functionalized MSNs were washed and dried for the following use.

### Synthesis and Characterization of Carboxymethyl Functionalized Inulin:

Carboxymethyl inulin (abbreviated as CMI) was prepared according to the method described by Sun.^{[18a][18a]} More specifically, 3.4 g of inulin was dissolved in 40 ml deionized water. Subsequently, 6.4 g NaOH and 7.5 g monochloroacetic acid (MCA) were added, respectively. After 5 h of stirring at 80° C., the reaction mixture was neutralized by 6M HCl solution and then concentrated. The resulting product was dialyzed in water using 500-1,000 molecular weight cut off dialysis bags for 24 h with the water changed twice in the 24 h period. The final solution was freeze dried.

The product was subsequently characterized by UV-vis, and FT-IR spectrometry. Infrared spectra were recorded as neat samples on a Nicolet iS20 FT-IR spectrometer (Thermo Fisher Scientific). UV-vis were recorded on a Beckman Coulter DU-800 spectrophotometer.

Preparation of Budesonide (Bud)-Loaded MSN-IN NPs: To synthesize the MSN-IN conjugate, NHS and EDC were used to prepare amine-reactive esters of the carboxylate groups located on the CMI to react with the amino groups on the surface of MSNs. Specifically, CMI was dissolved in 40 ml distilled water, into which NHS and EDC was added and magnetically stirred for 1 h at 37° C. Then, MSN-NH₂ suspended aqueous solution (10 mL) was added at a 1:1 weight equivalence and stirred overnight at 37° C. After completion, the reaction mixture was washed three times via centrifugal filtration at 12,000 rpm for 20 min. The prepared MSN-IN NPs were then collected, vacuum dried and stored at room temperature for the following use.

For the loading of budesonide, 100 mg of MSN-NH₂ and budesonide (100 mg) were suspended in dry acetonitrile (20 mL) under an inert atmosphere. The mixture was then stirred for 24 h at 60° C. to achieve maximum loading in the MCM-41 scaffolding pores. Furthermore, the drug-loaded MSNs were recovered by centrifugation at 12,000 rpm for 30 min and washed several times with ethanol to remove the free drug. Finally, Bud@MSN-IN nanoparticles were synthesized according to the above method.

### Characterization of the Nanoparticles

The nanoparticles were subsequently characterized by transmission electronic microscopy (TEM), N₂ adsorption-desorption isotherms, thermogravimetric analysis (TGA), UV-vis, and FT-IR spectrometry. The size and morphology of the prepared nanoparticles were characterized by a Hitachi HT7700 transmission electron microscopy (TEM) (Hitachi HT7700, Japan). UV-Vis-NIR absorption spectra were recorded using a Beckman Coulter DU-800 system with a slit width of 2.0 nm and a time constant of 1.0 s. Infrared spectra were recorded as neat samples on a Nicolet iS20 FT-IR spectrometer (Thermo Fisher Scientific). The N₂ adsorption-desorption isotherms were recorded with a Micrometrics ASAP2010 automated sorption analyzer. Samples were degassed at 120° C. in vacuum overnight. The Brunauer-Ummett-Teller (BET) model was applied to determine the specific surface areas using the absorption data in the low pressure range. Pore size was estimated following the Barrett, Joyner, and Halenda (BJH) method. TGA analysis was carried out on a simultaneous thermal analyzer, Netzsch STA449F3 TG-Differential Scanning calorimetry (DSC), between room temperature to 800° C. at a heating rate of 10° C./min under the nitrogen atmosphere. The hydrodynamic diameter distribution and zeta potentials of the nanoparticles were performed via a Malvern Zetasizer (Nano series ZS, UK; a dynamic Light Scattering and Zeta Potential measurement system) at room temperature. Data reported for size and zeta potential are the averages of three technical replicate runs. To favor the particle dispersion, samples were sonicated at 15 W for 2 min in each treatment until stabilization of the particle size distribution.

### Drug Loading Efficiency

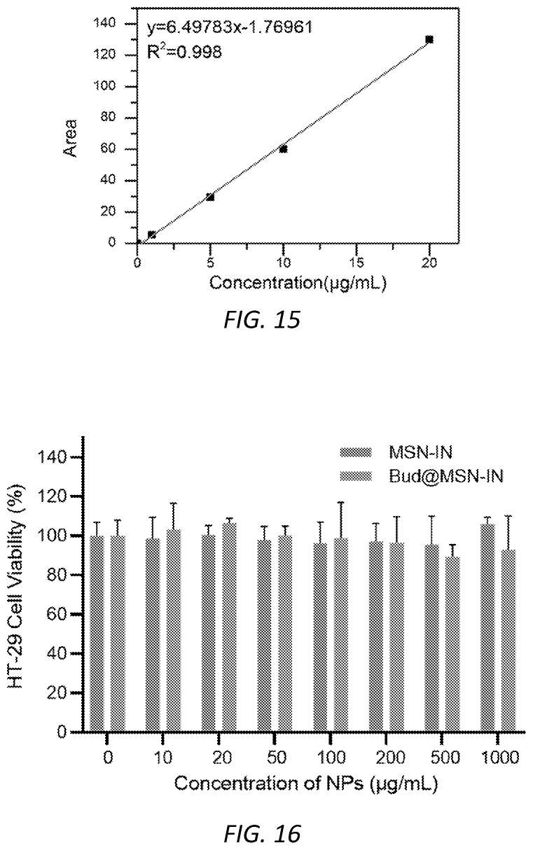

High-performance liquid chromatography (HPLC) (Agilent, Mississauga, Ontario, Canada) method was used to determine the concentrations of budesonide in the suspension. A HPLC Capcellpak C18 column (4.6× 250 mm, pore size 5 μm) (Merck KGaA, Germany) was used to perform the isocratic elution with a mobile phase consisting of methanol and distilled water (80:20, v/v), the flow rate of 0.8 mL/min, and column temperature of 40° C. Injection volume was 20 μL and budesonide content was measured at 240 nm. The method was linear (R²=0.998) over a range of 0 μg/mL to 20 μg/mL (FIG. 15).

The loading efficiency for triplicate samples was calculated, and the results were averaged using the following formula:

$$\% Loading efficiency = \frac{the weight of drug loaded in nanoparticles}{the weight of nanoparticles solid mass} \times 100 \% .$$

### In Vitro Drug Release Study in Simulated GI Fluids

The prepared nanoparticles were evaluated for efficacy with respect to targeting and drug release in the colon using an in vitro release assay that simulated gastrointestinal conditions. The NPs were first incubated in simulated gastric fluid (SGF) for 2 h, then incubated in simulated intestinal fluid (SIF) for 4 h and then incubated in simulated colonic fluid (SCF) for more than 18 hours in order to simulate gastrointestinal (GI) conditions. SGF (pH 1.2, pepsin 0.32%, w/v) and SIF (pH 6.8, pancreatin 1%, w/v) were prepared according to United State Pharmacopoeia 25 NF 20; whereas, SCF was composed of 0.1 M potassium phosphate buffer (pH 7.4) and inulinase (5 U/mL). Next, 0.1 g Bud@MSN-IN nanoparticles dry powders were dissolved in a 20 mL simulation medium and stirred at 37° C. At designated time intervals, 4 mL of release medium was taken out and then centrifuged at 13,000 rpm for 20 min. Subsequently, the precipitate and 4 mL fresh simulation fluid were supplemented into the simulation system. The cumulative drug release from the supernatant was quantified by HPLC at 240 nm.

### MTT Assay

HT-29 cells were cultured in DMEM medium supplemented with 10% (v/v) FBS, 100 U/mL of penicillin, and 100 μg/mL of streptomycin in a 5% CO₂ humidified environment at 37° C. For the methyl thiazolyl tetrazolium (MTT) assay, cells were seeded on 96-well plates at a density of 5,000 cells per well and were cultured for 24 h. Subsequently, cells were treated with the medium containing budesonide suspension (BDS-sus), MSN-IN NPs or Bud@MSN-IN NPs (with or without inulinase) at various concentrations for 24 h. After incubation, cells were washed with fresh culture medium and 100 μL of fresh culture medium was added to each well, followed by the addition of 10 μL of MTT (5 mg/mL) and, after incubation for 1 h at 37° C., the absorbance was measured at 490 nm using a 96-well plate microreader.

### Anti-Oxidative Activity of Bud@MSN-IN NPs in HT-29 Cells

HT-29 cells (5× 104/well) were plated into glass petri dishes and cultured in the medium for 24 h. Then, the medium was changed and cells were treated with different materials for 24 h. Cells without treatment were used as a control group. Diacetyldichlorofluorescein (DCFH-DA) was used as an intracellular reactive oxygen species (ROS) probe. Viable cells were stained with calcein-AM, while dead/apoptotic cells were stained with propidium iodide (PI). The fluorescence was determined using excitation/emission filters at 488/546 nm and a Synergy HT multidetection microplate reader.

### Animals

Female C57-BL/6 mice (6-8 weeks, 18-25 g) were obtained from Vital River Laboratories and were kept in standard conditions with humidity maintained at 50-60%, temperature controlled at 25±2° C., a 12-h dark/light cycle and free access to water and food. Mice were co-housed for a week before random assignment to experimental groups in order to synchronize the gut microbiome and reduce heterogeneity in the microbiota of individual mice.[43][43]

### DSS-Induced Model of Colitis and Drug Administration

Female C57-BL/6 mice were acclimatized for 1 week before inclusion in the study. Acute colitis was induced by feeding mice drinking water containing 3% (w/v) dextran sulfate sodium salt (DSS; MW 40000; Aladdin) ad libitum, starting from day 0 for 5 days, followed by changing to regular drinking water for an additional 5 days. The mice were randomly assigned into 5 groups: control group (Control), DSS-induced colitis group (Colitis), free budesonide suspension-treated group (Bud-sus), MSN-IN nanoparticles-treated group (MSN-IN NPs), as well as Bud@MSN-IN nanoparticles-treated group (Bud@MSN-IN NPs). 0.6 mg/kg of Budesonide or an equal amount of nanoparticles suspension was orally administered daily to the different groups during 10 days experimental period. On the last day of the experiment, mice were sacrificed and the entire colon was excised. Colon length was measured and gently washed with physiological saline. Then, two pieces of 0.5 cm of the distal section were used for histological assessment and immunofluorescence staining, respectively. The remaining colon tissues were excised for histological evaluation and ELISA analysis.

### Histology Analysis and Immunofluorescence Staining

Fresh colon tissues were fixed with 10% formalin for more than 24 h, followed by embedding with paraffin and division into full thick sections of 5 μm. Subsequently, hematoxylin and eosin (H&E) were applied for staining of the sections to characterize histological changes, which was observed by a Nikon ECLIPSE TI-SR (Tokyo, Japan) fluorescence microscope. Histological sections were scored as described previously.[18a][18a] In brief, for the epithelium (E), the standards were as follows: 0, normal morphology; 1, loss of goblet cells; 2, loss of goblet cells in large areas; 3, loss of crypts; and 4, loss of crypts in large areas. For the infiltration (I), it was evaluated by the following standard scores: 0, no infiltrate; 1, infiltrate around the crypt basis; 2, infiltrate reaching the muscularis mucosae; 3, extensive infiltration reaching the muscularis mucosae and thickening of the mucosa with abundant edema; and 4, infiltration of the submucosa. The total histological score was presented as E+I.

For immunofluorescence staining, colonic tissue sections were deparaffinized and rehydrated with xylene, 100% and 95% ethanol, and antigen retrieved through a heat-induced antigen retrieval method in 10 mM sodium citrate buffer (pH 6.0). The slides were then blocked with 10% goat serum for 30 min at 37° C. and incubated with primary antibodies as follows: ZO-1 rabbit polyclonal antibody (cat. no. 61-7300, 1:100, Invitrogen) together overnight at 4° C. The second antibody against mouse was conjugated with anti-rabbit antibody with Alexa fluor-488 for 1 h at 37° C. The Occludin-1 antibody (OC-3F10) (cat. no. 33-1500, 1:100, Invitrogen) was conjugated with AlexaFluor-594. Nuclei were counterstained with DAPI (4′, 6-diamidino-2-phenylindole) for 3 min. Images were captured using a fluorescence microscope (Fluoview FV1000, Olymups, Japan).

### Assessment of the Disease Activity Index

During the whole period of treatment, changes in the body weight, visible stool consistency, and fecal bleeding were assessed each day. Disease activity index (DAI) was the combined scores of weight loss, stool consistency and bleeding divided by three. Stool consistency index was determined as follows: 0=normal; 1=loose stool; 2=mild diarrhea; 3=diarrhea; 4=gross diarrhea. Fecal bleeding index was assessed according to the following criteria: 0 none; 1-Occult bleeding in stool; 2=blood traced in stool visible; 3=obvious blood in stool; 4-totally rectal bleeding. Weight loss index was determined as follows: 0=no change; 1=≤5%; 2=6-10%; 3=11-20%; 4=≥20%.[44][44]

### ELISA Assay

The colonic length was measured and gently washed with saline to remove the contents. Then, 1 cm of the distal section was used for histological assessment. The remaining section was broken by a tissue disrupter and centrifuged to collect tissue supernatant. Then the levels of interferon-γ (IFN-γ), interleukin-6 (IL-6), interleukin-1B (IL-1B), as well as myeloperoxidase (MPO) activity were measured by ELISA.

### Gut Microbiota Analysis

Total DNA was extracted from fecal samples using the E.Z.N.A.® Stool DNA Kit (D4015, Omega, Inc., USA) according to manufacturer ‘s instructions. The extracted DNA was then used as a template to amplify the V3-V4 regions of 16S rRNA genes using the universal primers 341F (5’-CCTACGGGNGGCWGCAG-3′) and 805R (5′-GACTACHVGGGTATCTAATCC-3′) [45][45] where N=any nucleobase (A, T, C, or G); W=A or T; H=A, T, or C; and V=A, C, or G. PCR amplification was performed in a total volume of 25 μL reaction mixture containing 25 ng of template DNA, 12.5 μL PCR Premix, 2.5 μL of each primer, and PCR-grade water to adjust the volume. The PCR conditions to amplify the prokaryotic 16S fragments including: 98° C. for 30 seconds(s), followed by 32 cycles at 98° C. for 10 s, 54° C. for 30 s and 72° C. for 45 s, where the final extension step was performed at 72° C. for 10 min. The PCR products were confirmed with 2% agarose gel electrophoresis. The PCR products were purified by AMPure XT beads (Beckman Coulter Genomics, Danvers, MA, USA) and quantified by a Qubit fluorometer (Invitrogen, USA). The completed library was sequenced using the Illumina NovaSeq PE250 platform, a high-throughput sequencing platform, (according to the manufacturer's recommendations) provided by LC-Bio. Paired-end reads from the original DNA fragments were merged using FLASh (Fast Length Adjustment of SHort reads). Alpha diversity and beta diversity were calculated by normalized to the same sequences randomly. Then according to SILVA (release 132) classifier, feature abundance was normalized using relative abundance of each sample. Beta diversity was calculated by QIIME and the graphs were drawn by R package (v3.5.2). Blast was used for sequence alignment, and the feature sequences were annotated with SILVA database for each representative sequence.

### In Vivo Biocompatibility Study

To evaluate the in vivo toxicity of the MSN-IN and Bud@MSN-IN NPs via oral administration, female C57BL/6 mice were assigned randomly to three groups: the untreated group; the MSN-IN group, normal mice orally treated with MSN-IN NPs (20 mg/kg) daily; and the Bud@MSN-IN group, normal mice orally treated with Bud@MSN-IN NPs (20 mg/kg) daily. Post 10 days of administration, animals were euthanized, and blood samples were collected for hematological analysis and quantification of biochemical markers relevant to liver/kidney functions. Major organs (heart, kidneys, liver, lung, and spleen) were prepared and H&E-stained for histological evaluations. Untreated normal mice were used as a control.

The excretion kinetics of Bud@MSN-IN NPs was investigated in healthy female C57-BL/6 mice, including 4 groups: a control, a 1 day, a 2 days, and a 3 days group. At 1 day, 2 days, and 3 days following a single oral administration of Bud@MSN-IN NPs (20 mg/kg), the mice were sacrificed and the brain, heart, liver, spleen, lung, kidney, stomach, small intestine, and colon were collected. The mass of each tissue was recorded. Next, the tissue samples were transferred to tubes and the silica concentration in tissue samples was analyzed using Inductively Coupled Plasma Mass Spectrometry (ICP-MS) after dissolving the sample completely using concentrated nitric acid.

### Statistical Analysis

All statistical analyses were performed using GraphPad Prism 8.0.2 (California, USA). Data are expressed as mean±SD (standard deviation). A one-way or two-way ANOVA, followed by Tukey's HSD multiple comparison post hoc test was used for testing differences among groups. A value of P<0.05 was considered statistically significant.

### REFERENCES

[1] a) B. Khor, A. Gardet, R. J. Xavier, Nature 2011, 474, 307; b) S. C. Ng, H. Y. Shi, N. Hamidi, F. E. Underwood, W. Tang, E. I. Benchimol, R. Panaccione, S. Ghosh, J. C. Y. Wu, F. K. L. Chan, J. J. Y. Sung, G. G. Kaplan, Lancet 2017, 390, 2769.
[2] a) T. Kucharzik, P. Ellul, T. Greuter, J. F. Rahier, B. Verstockt, C. Abreu, A. Albuquerque, M. Allocca, M. Esteve, F. A. Farraye, H. Gordon, K. Karmiris, U. Kopylov, J. Kirchgesner, E. MacMahon, F. Magro, C. Maaser, L. de Ridder, C. Taxonera, M. Toruner, L. Tremblay, M. Scharl, N. Viget, Y. Zabana, S. Vavricka, J Crohns Colitis 2021, 15, 879; b) C. A. Lamb, N. A. Kennedy, T. Raine, P. A. Hendy, P. J. Smith, J. K. Limdi, B. Hayee, M. C. E. Lomer, G. C. Parkes, C. Selinger, K. J. Barrett, R. J. Davies, C. Bennett, S. Gittens, M. G. Dunlop, O. Faiz, A. Fraser, V. Garrick, P. D. Johnston, M. Parkes, J. Sanderson, H. Terry, I. B. D. g. e. c. group, D. R. Gaya, T. H. Iqbal, S. A. Taylor, M. Smith, M. Brookes, R. Hansen, A. B. Hawthorne, Gut 2019, 68, s1.
[3] J. P. Gisbert, A. C. Marin, M. Chaparro, Am J Gastroenterol 2016, 111, 632; b) R. P. Hirten, M. Iacucci, S. Shah, S. Ghosh, J. F. Colombel, Clin Gastroenterol Hepatol 2018, 16, 1374.
[4] a) R. Caruso, B. C. Lo, G. Nunez, Nat Rev Immunol 2020, 20, 411; b) J. Y. Lee, R. M. Tsolis, A. J. Baumler, Science 2022, 377, eabp9960.
[5] Y. Zhang, T. Wang, M. Sun, Y. Song, X. Huang, S. Zhang, J. Yang, J. Zhang, Adv Healthc Mater 2023, 12, e2300069.
[6] a) Y. Zhang, L. Wang, Z. D. Wang, Q. Zhou, X. Zhou, T. Zhou, Y. X. Guan, X. Liu, J Nanobiotechnology 2023, 21, 145; b) L. E. McCoubrey, A. Favaron, A. Awad, M. Orlu, S. Gaisford, A. W. Basit, J Control Release 2023, 353, 1107; c) R. Han, H. He, Y. Lu, H. Lu, S. Shen, W. Wu, J Control Release 2024, 370, 256.
[7] A. Lamprecht, H. Yamamoto, H. Takeuchi, Y. Kawashima, J Control Release 2005, 104, 337.
[8] P. S. Yavvari, P. Verma, S. A. Mustfa, S. Pal, S. Kumar, A. K. Awasthi, V. Ahuja, C. V. Srikanth, A. Srivastava, A. Bajaj, Nanoscale 2019, 11, 4970.
[9] T. Florin, R. Movva, J. Begun, J. Duley, I. Oancea, P. O. Cuiv, Gut Microbes 2018, 9, 175.
[10] a) V. C. Ibekwe, H. M. Fadda, G. E. Parsons, A. W. Basit, Int J Pharm 2006, 308, 52; b) A. Awad, C. M. Madla, L. E. McCoubrey, F. Ferraro, F. K. H. Gavins, A. Buanz, S. Gaisford, M. Orlu, F. Siepmann, J. Siepmann, A. W. Basit, Adv Drug Deliv Rev 2022, 181, 114076.
[11] G. B. Hatton, C. M. Madla, S. C. Rabbie, A. W. Basit, Int J Pharm 2018, 548, 408.
[12] K. R. James, T. Gomes, R. Elmentaite, N. Kumar, E. L. Gulliver, H. W. King, M. D. Stares, B. R. Bareham, J. R. Ferdinand, V. N. Petrova, K. Polanski, S. C. Forster, L. B. Jarvis, O. Suchanek, S. Howlett, L. K. James, J. L. Jones, K. B. Meyer, M. R. Clatworthy, K. Saeb-Parsy, T. D. Lawley, S. A. Teichmann, Nat Immunol 2020, 21, 343.
[13] I. Ferrocino, V. Ponzo, M. Pellegrini, I. Goitre, M. Papurello, I. Franciosa, C. D'Eusebio, E. Ghigo, L. Cocolin, S. Bo, Sci Rep 2022, 12, 9192.
[14] D. T. Rubin, A. N. Ananthakrishnan, C. A. Siegel, B. G. Sauer, M. D. Long, Am J Gastroenterol 2019, 114, 384.
[15] a) R. Hamedani, R. D. Feldman, B. G. Feagan, Aliment Pharmacol Ther 1997, 11 Suppl 3, 98; b) S. S. Seoudi, E. A. Allam, A. H. El-Kamel, H. Elkafrawy, R. M. El-Moslemany, Drug Deliv Transl Res 2023, 13, 2930.
[16] a) C. W. Ang, L. Tan, Z. Qu, N. P. West, M. A. Cooper, A. Popat, M. A. T. Blaskovich, ACS Biomater Sci Eng 2022, 8, 4196; b) Q. Li, W. Liu, K. Liu, Z. Dong, W. Kong, X. Lu, Y. Wei, W. Wu, J. Yang, J. Qi, ACS Appl Mater Interfaces 2024, 16, 33081.
[17] M. Usman, C. Zhang, P. J. Patil, A. Mehmood, X. Li, M. Bilal, J. Haider, S. Ahmad, Carbohydr Polym 2021, 252, 117176.
[18] a) Q. Sun, L. Luan, M. Arif, J. Li, Q. J. Dong, Y. Gao, Z. Chi, C. G. Liu, Carbohydr Polym 2018, 189, 352; b) B. N. Limketkai, G. Godoy-Brewer, N. D. Shah, L. Maas, J. White, A. M. Parian, G. E. Mullin, Inflamm Bowel Dis 2024.
[19] a) D. Lopez-Molina, S. Chazarra, C. W. How, N. Pruidze, E. Navarro-Peran, F. Garcia-Canovas, P. A. Garcia-Ruiz, F. Rojas-Melgarejo, J. N. Rodriguez-Lopez, Int J Pharm 2015, 479, 96; b) K. Kourani, P. Jain, A. Kumar, A. K. Jangid, G. Swaminathan, V. R. Durgempudi, J. Jose, R. Reddy, D. Pooja, H. Kulhari, L. D. Kumar, Nanomedicine: nanotechnology, biology, and medicine 2022, 40, 102504; c) Y. Hou, J. Jin, H. Duan, C. Liu, L. Chen, W. Huang, Z. Gao, M. Jin, Biomaterials 2022, 283, 121440.
[20] a) V. Tremaroli, F. Backhed, Nature 2012, 489, 242; b) Y. Li, K. J. Hintze, R. E. Ward, Biochimie 2021, 185, 43; c) Z. Liu, F. Liu, W. Wang, C. Sun, D. Gao, J. Ma, M. A. Hussain, C. Xu, Z. Jiang, J. Hou, Food Funct 2020, 11, 3823; d) Y. Du, K. Kusama, K. Hama, X. Chen, Y. Tahara, S. Kajiwara, S. Shibata, K. Orihara, Int J Mol Sci 2024, 25.
[21] L. Santiago-Rodriguez, M. M. Lafontaine, C. Castro, J. Mendez-Vega, M. Latorre-Esteves, E. J. Juan, E. Mora, M. Torres-Lugo, C. Rinaldi, J Mater Chem B 2013, 1, 2807.
[22] Y. Mi, J. Zhang, X. Han, W. Tan, Q. Miao, J. Cui, Q. Li, Z. Guo, Int J Biol Macromol 2021, 181, 572.
[23] S. Vaidyanath, B. S. Harish, G. Gayathri, C. Trilokesh, K. B. Uppuluri, V. Anbazhagan, Int J Biol Macromol 2020, 160, 964.
[24] B. Hufnagel, V. Muellner, K. Hlatky, C. Tallian, R. Vielnascher, G. M. Guebitz, M. Wirth, F. Gabor, Carbohydr Polym 2021, 252, 117091.
[25] B. Chami, N. J. J. Martin, J. M. Dennis, P. K. Witting, Arch Biochem Biophys 2018, 645, 61.
[26] C. Li, Y. Zhao, J. Cheng, J. Guo, Q. Zhang, X. Zhang, J. Ren, F. Wang, J. Huang, H. Hu, R. Wang, J. Zhang, Adv Sci (Weinh) 2019, 6, 1900610.
[27] a) F. Chen, G. Huang, Int J Biol Macromol 2019, 141, 14; b) L. Huang, M. Huang, M. Shen, P. Wen, T. Wu, Y. Hong, Q. Yu, Y. Chen, J. Xie, Int J Biol Macromol 2019, 136, 1000.
[28] S. Stoyanova, J. Geuns, E. Hideg, W. Van den Ende, Int J Food Sci Nutr 2011, 62, 207.
[29] a) B. Hu, S. Yu, C. Shi, J. Gu, Y. Shao, Q. Chen, Y. Li, R. Mezzenga, ACS Nano 2020, 14, 2760; b) L. Su, L. Shen, D. R. Clayburgh, S. C. Nalle, E. A. Sullivan, J. B. Meddings, C. Abraham, J. R. Turner, Gastroenterology 2009, 136, 551.
[30] a) S. Citi, Science 2018, 359, 1097; b) Y. Lee, K. Sugihara, M. G. Gillilland, 3rd, S. Jon, N. Kamada, J. J. Moon, Nat Mater 2020, 19, 118.
[31] a) J. Yang, M. Peng, S. Tan, S. Ge, L. Xie, T. Zhou, W. Liu, K. Zhang, Z. Zhang, J. Liu, J. Shi, ACS Cent Sci 2023, 9, 1327; b) W. F. Song, W. Q. Yao, Q. W. Chen, D. Zheng, Z. Y. Han, X. Z. Zhang, ACS Cent Sci 2022, 8, 1306.
[32] a) J. Lloyd-Price, C. Arze, A. N. Ananthakrishnan, M. Schirmer, J. Avila-Pacheco, T. W. Poon, E. Andrews, N. J. Ajami, K. S. Bonham, C. J. Brislawn, D. Casero, H. Courtney, A. Gonzalez, T. G. Graeber, A. B. Hall, K. Lake, C. J. Landers, H. Mallick, D. R. Plichta, M. Prasad, G. Rahnavard, J. Sauk, D. Shungin, Y. Vazquez-Baeza, R. A. White, 3rd, I. Investigators, J. Braun, L. A. Denson, J. K. Jansson, R. Knight, S. Kugathasan, D. P. B. McGovern, J. F. Petrosino, T. S. Stappenbeck, H. S. Winter, C. B. Clish, E. A. Franzosa, H. Vlamakis, R. J. Xavier, C. Huttenhower, Nature 2019, 569, 655; b) A. D. Kostic, R. J. Xavier, D. Gevers, Gastroenterology 2014, 146, 1489.
[33] L. Zhao, X. Du, J. Tian, X. Kang, Y. Li, W. Dai, D. Li, S. Zhang, C. Li, Front Pharmacol 2021, 12, 644387.
[34] a) N. R. Shin, T. W. Whon, J. W. Bae, Trends Biotechnol 2015, 33, 496; b) D. N. Frank, A. L. St Amand, R. A. Feldman, E. C. Boedeker, N. Harpaz, N. R. Pace, Proc Natl Acad Sci USA 2007, 104, 13780.
[35] W. Zhu, M. G. Winter, M. X. Byndloss, L. Spiga, B. A. Duerkop, E. R. Hughes, L. Buttner, E. de Lima Romao, C. L. Behrendt, C. A. Lopez, L. Sifuentes-Dominguez, K. Huff-Hardy, R. P. Wilson, C. C. Gillis, C. Tukel, A. Y. Koh, E. Burstein, L. V. Hooper, A. J. Baumler, S. E. Winter, Nature 2018, 553, 208.
[36] K. L. Madsen, J. S. Doyle, L. D. Jewell, M. M. Tavernini, R. N. Fedorak, Gastroenterology 1999, 116, 1107.
[37] S. Oliva, G. Di Nardo, F. Ferrari, S. Mallardo, P. Rossi, G. Patrizi, S. Cucchiara, L. Stronati, Aliment Pharmacol Ther 2012, 35, 327.
[38] Z. Zhang, X. Wu, S. Cao, M. Cromie, Y. Shen, Y. Feng, H. Yang, L. Li, Nutrients 2017, 9.
[39] S. K. Jain, A. Jain, Expert Opin Drug Deliv 2008, 5, 483.
[40] N. Shah, T. Shah, A. Amin, Expert Opin Drug Deliv 2011, 8, 779.
[41] a) M. Roldo, E. Barbu, J. F. Brown, D. W. Laight, J. D. Smart, J. Tsibouklis, Expert Opin Drug Deliv 2007, 4, 547; b) S. Zhang, R. Langer, G. Traverso, Nano Today 2017, 16, 82.
[42] X. Chen, H. Sun, J. Hu, X. Han, H. Liu, Y. Hu, Colloids Surf B Biointerfaces 2017, 152, 77.
[43] K. D. McCoy, M. B. Geuking, F. Ronchi, Curr Protoc Immunol 2017, 117, 23 1 1.
[44] S. Cheng, H. Shen, S. Zhao, Y. Zhang, H. Xu, L. Wang, B. Di, L. Xu, C. Hu, Nanoscale 2020, 12, 15348.
[45] J. B. Logue, C. A. Stedmon, A. M. Kellerman, N. J. Nielsen, A. F. Andersson, H. Laudon, E. S. Lindstrom, E. S. Kritzberg, ISME J 2016, 10, 533.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising:
a mesoporous silica nanoparticle (MSN), and
inulin conjugated to the surface of the MSN.

2. The composition of claim 1, comprising at least one bioactive agent loaded into the MSN, optionally comprising at least two bioactive agents loaded into the MSN.

3. The composition of claim 1, wherein the inulin comprises at least one glycosidic bond.

4. The composition of claim 1, wherein the inulin is sensitive to enzymatic degradation.

5. The composition of claim 4, wherein the inulin is sensitive to degradation by inulinase.

6. The composition of claim 1, wherein the inulin comprises carboxymethyl inulin (CMI).

7. The composition of claim 2, wherein the at least one bioactive agent is selected from the group consisting of a small molecule, an organometallic compound, a vaccine, a diagnostic agent, a polynucleotide, a peptide, and a polypeptide.

8. The composition of claim 1, wherein the MSN comprises at least one pore on its surface.

9. The composition of claim 8, wherein the at least one pore comprises a diameter ranging from about 2 nm to about 50 nm.

10. The composition of claim 1, wherein the composition comprises a particle size ranging from about 100 nm to about 500 nm in diameter, optionally wherein the composition comprises a particle size of about 275 nm in diameter.

11. The composition of claim 1, wherein the composition comprises a zeta potential ranging from about −20 mV to about −10 mV.

12. A pharmaceutical composition comprising the composition of claim 2 and at least one pharmaceutically accepted carrier.

13. The pharmaceutical composition of claim 12, wherein the pharmaceutical composition comprises an oral formulation.

14. The pharmaceutical composition of claim 13, wherein the oral formulation is selected from the group consisting of tablets, pills, capsules, powders, granules, solutions, and suspensions.

15. A method of administering to a subject in need thereof the pharmaceutical composition of claim 12, wherein the subject in need thereof is suspected of having or has been diagnosed as having a colonic disease or disorder.

16. The method of claim 15, wherein less than about 10% of the at least one bioactive agent is released from the composition in the subject's upper GI track after administration.

17. The method of claim 15, wherein more than about 60% of the at least one bioactive agent is released from the composition in the subject's colon after administration.

18. A method for treating a colonic disease or disorder, comprising administering to a subject in need thereof a therapeutically effective amount of the pharmaceutical composition of claim 12, wherein the subject in need thereof is suspected of having or has been diagnosed as having a colonic disease or disorder.

19. The method of claim 18, wherein the therapeutically effective amount of the pharmaceutical composition is administered in combination with at least one biological agent.

20. A method of modulating a composition of gut microbiome in a subject in need thereof comprising administering to the subject in need thereof a therapeutically effective amount of the composition of claim 1, wherein the subject in need thereof is suspected of having or has been diagnosed as having a colonic disease or disorder, optionally wherein the composition comprises at least one bioactive agent loaded into the MSN.

21. The method of claim 20, wherein at least one bacteria in fecal microbiota of the subject is increased following administration, optionally wherein the at least one bacteria is selected from the group consisting of Firmicutes, Bacteroidetes, Verrucomicrobia, Alloprevotella, Lactobacillus, Akkermansia, Prevotellaceae, and Proteobacteria phyla.

22. The method of claim 21, wherein the amount of Alloprevotella, Lactobacillus, Akkermansia, Prevotellaceae, or any combination thereof is increased in fecal microbiota of the subject following administration.

23. The method of claim 15, wherein the colonic disease or disorder is selected from the group consisting of colorectal cancer, colonic polyps, Crohn's disease, diverticulitis, inflammatory bowel disease (IBD), irritable bowel syndrome (IBS), microscopic colitis, and ulcerative colitis (UC).

24. The method of claim 23, wherein the colonic disease or disorder is IBD.
